(19) United States
(12) Patent Application Publication
(10) Pub. No.: US 2024/0327809 A1
(43) Pub. Date: Oct. 3, 2024

(54) Enzymes and Uses Thereof

(71) Applicant: Samsara Eco Pty Limited, Crows Nest (AU)

(72) Inventors: Colin John Jackson, Crows Nest (AU); Vanessa Vongsouthi, Crows Nest (AU); Matthew Spence, Crows Nest (AU)

(21) Appl. No.: 18/578,768

(22) PCT Filed: Jul. 15, 2022
(86) PCT No.: PCT/AU2022/050745

§ 371 Date: Jan. 12, 2024

(30) Foreign Application Priority Data
Jul. 15, 2021 (AU) 2021902167

(51) Int. Cl. C12N 9/18; C07C 29/147; C07C 51/09; C08J 11/10; C12P 7/18; C12P 7/44

(52) U.S. Cl. CPC C12N 9/18; C07C 29/147; C07C 51/09; C08J 11/105; C12P 7/18; C12P 7/44; C12Y 301/01074; C08J 2367/02

(57) ABSTRACT

The present disclosure relates generally to a polypeptide having esterase activity, more specifically, to a polypeptide comprising an amino acid sequence that has at least 80% identity to amino acids 2-257 of SEQ ID NO: 1.

The present invention relates to novel synthetic enzymes, more particularly to recombinant enzymes that catalyse the hydrolysis of mono- and di-, and poly-terephthalic acid esters and uses thereof.

### BACKGROUND

All references, including any patent or patent application cited in this specification are hereby incorporated by reference to enable full understanding of the invention. Nevertheless, such references are not to be read as constituting an admission that any of these documents forms part of the common general knowledge in the art, in Australia or in any other country.

Global industrialization has had significant environmental impact, not least of which is an increase in the manufacture and reliance on plastic and plastic products. Whilst there is a growing effort to find suitable and environmentally sustainable alternatives to plastics, including their manufacture and disposal, such products remain a significant problem and contribute to a vast majority of environmental pollutants. One of the major contributors to this problem is polyethylene terephthalate (PET) and its waste products, millions of tons of which are produced globally every year. The environmental significance of this problem is attributed, at least in part, to the chemical nature of plastics, in particular PET based products, as they do not readily decompose in nature.

Approaches to deal with the problem of plastic waste products have typically included incineration, disposal in landfill and mechanical disintegration. However, these approaches also have significant environmental impact. For instance, incineration of plastics produces potentially harmful byproducts that are released into the atmosphere; the decomposition rate of plastics in landfill is typically very slow and there is a risk that toxic materials will leach into groundwater; and mechanical disintegration is relatively expensive and inefficient and there is often limited use for its byproducts.

More recently, biological (enzymatic) degradation of plastics has been considered as an alternative approach to reducing plastic waste accumulation. This approach includes the use of PETases, an esterase class of enzyme that catalyze the hydrolysis of PET to the monomeric mono-2-hydroxyethyl terephthalate (MHET), and the resultant MHET is further broken down by the action of MHETases to terephthalic acid and ethylene glycol. Terephthalic acid is the precursor to polyester PET.

Whilst enzymatic degradation of plastics is an attractive alternative to alleviating the environmental impact of plastic waste products and their disposal, it has not yet seen widespread adoption, including because of its relative inefficiency, slow rate of enzymatic degradation and low levels of enzyme expression in common industrial host organisms. Hence, there remains an urgent need for improved methods and reagents for the enzymatic degradation of plastics.

### SUMMARY OF THE INVENTION

In one aspect disclosed herein, there is provided a polypeptide having esterase activity, wherein the polypeptide comprises an amino acid sequence that has at least 80% identity to amino acids 2-257 of SEQ ID NO:1.

In embodiments disclosed herein, there is provided polypeptides that comprise an amino acid sequence that differs from SEQ ID NO:1 by an amino acid substitution at one or more positions corresponding to amino acid position selected from the group consisting of:
i. a position that corresponds to amino acid position 7 of SEQ ID NO:1;
ii. a position that corresponds to amino acid position 10 of SEQ ID NO:1;
iii. a position that corresponds to amino acid position 11 of SEQ ID NO:1;
iv. a position that corresponds to amino acid position 13 of SEQ ID NO:1;
v. a position that corresponds to amino acid position 24 of SEQ ID NO:1;
vi. a position that corresponds to amino acid position 32 of SEQ ID NO:1;
vii. a position that corresponds to amino acid position 45 of SEQ ID NO:1;
viii. a position that corresponds to amino acid position 48 of SEQ ID NO:1;
ix. a position that corresponds to amino acid position 51 of SEQ ID NO:1;
x. a position that corresponds to amino acid position 72 of SEQ ID NO:1;
xi. a position that corresponds to amino acid position 87 of SEQ ID NO:1;
xii. a position that corresponds to amino acid position 90 of SEQ ID NO:1;
xiii. a position that corresponds to amino acid position 105 of SEQ ID NO:1;
xiv. a position that corresponds to amino acid position 119 of SEQ ID NO:1;
xv. a position that corresponds to amino acid position 158 of SEQ ID NO:1;
xvi. a position that corresponds to amino acid position 192 of SEQ ID NO:1;
xvii. a position that corresponds to amino acid position 211 of SEQ ID NO:1;
xviii. a position that corresponds to amino acid position 222 of SEQ ID NO:1;
xix. a position that corresponds to amino acid position 235 of SEQ ID NO:1;
xx. a position that corresponds to amino acid position 243 of SEQ ID NO:1; and
xxi. a position that corresponds to amino acid position 246 of SEQ ID NO:1.

In an embodiment, the polypeptide further comprises an amino acid sequence that differs from SEQ ID NO:1 by an amino acid substitution at one or more positions corresponding to amino acid position selected from the group consisting of:
i. a position that corresponds to amino acid position 202 of SEQ ID NO:1;
ii. a position that corresponds to amino acid position 207 of SEQ ID NO:1; and
iii. a position that corresponds to amino acid position 249 of SEQ ID NO:1.

In an embodiment disclosed herein, the polypeptide comprises an amino acid sequence that differs from SEQ ID NO:1 by amino acid substitutions at positions corresponding to amino acid positions N202, F207 and S249 of SEQ ID NO:1. In another embodiment, the amino acid substitutions at the positions corresponding to amino acid positions N202, F207 and S249 of SEQ ID NO:1 are N202C, F207V and S249C.

In another embodiment disclosed herein, the polypeptide comprises an N-terminal cellular export signal peptide. In another embodiment, the N-terminal cellular export signal peptide comprises the amino acid sequence of SEQ ID NO:28 or SEQ ID NO:29. In a preferred embodiment, the N-terminal cellular export signal peptide comprises the amino acid sequence of SEQ ID NO:28.

The present disclosure also extends to a composition comprising the polypeptide as described herein.

The present disclosure also extends to a nucleic acid sequence encoding the polypeptide described herein.

The present disclosure also extends to an expression vector comprising the nucleic acid sequence described herein.

The present disclosure also extends to a host cell comprising the nucleic acid sequence or the expression vector described herein.

In another aspect, the present disclosure provides a method of producing a polypeptide having esterase activity, the method comprising:
i. providing the polynucleotide described herein;
ii. expressing the polynucleotide in a host cell under conditions sufficient to allow the host cell to produce the polypeptide; and
iii. collecting the polypeptide produced by the host cell in ii.

In yet another aspect, there is provided a method for hydrolysing a terephthalic acid ester, the method comprising contacting a terephthalic acid ester with the polypeptide, the composition, or the host cell described herein, under conditions sufficient to convert the terephthalic acid ester to terephthalic acid and ethylene glycol, mono-(2-hydroxyethyl) terephthalate and/or bis-(2-hydroxyethyl) terephthalate.

The present disclosure also extends to a method of degrading a plastic product comprising a polyester, the method comprising contacting the plastic product with the polypeptide, the composition, or the host cell described herein, under conditions sufficient to degrade the plastic product. In an embodiment, the plastic product comprises the polyester polyethylene terephthalate (PET).

In another embodiment, the methods disclosed herein comprise:
i. exposing the PET to the polypeptides, compositions or host cells disclosed herein under conditions sufficient for the polypeptides to catalyze the conversion of the PET to generate terephthalic acid, mono-(2-hydroxyethyl) terephthalate and/or bis-(2-hydroxyethyl) terephthalate; and
ii. simultaneously or sequentially, exposing the PET to
   a. a mono-(2-hydroxyethyl) terephthalate hydrolase (MHETase), or to an enzyme having MHETase activity;
   b. a wild-type or extant PETase; and/or
   c. a cutinase having PETase activity.

The present disclosure also extends to a composition comprising the terephthalic acid and/or ethylene glycol recovered by the method disclosed herein.

In another aspect, there is provided a host cell genetically modified to express the polypeptide described herein.

In another aspect, there is provided a method of producing a plastic product using the composition of the terephthalic acid and ethylene glycol generated by the method disclosed herein.

### BRIEF DESCRIPTION OF THE FIGURES

FIG. 1 shows the amino acid sequences of SEQ ID NO:1 and SEQ ID NO:2.

FIG. 2 shows the sequence alignment of the engineered polypeptide having esterase activity of SEQ ID NO:1 and its variants, with related extant cutinases with UniProt identification numbers E5BBQ3, E5BBQ2, E9LVH9 and G9BY57.

FIG. 3 shows the kinetic data of the engineered polypeptide having esterase activity of SEQ ID NO:1 and its variants, measured by the amount of product produced, observed as absorbance of the soluble products of hydrolysis (TPA, mono-2-hydroxyethyl terephthalate (MHET) and bis(2-hydroxyethyl) terephthalate (BHET)) within the range of 240-300 nm, after 16 h incubation with amorphous PET film at 60-70 degrees C.

FIG. 4 shows the activity of the engineered polypeptide having esterase activity of SEQ ID NO:1 and its variants, and the extant cutinases (LCC, Tfcut2, E9LV10, E9LV9, E5BBQ2) at 60 degrees C. A control reaction (no enzyme) is shown as a grey bar. All data are mean+/−SEM (n=2). Absorbance of the soluble products of hydrolysis (TPA, mono-2-hydroxyethyl terephthalate (MHET) and bis(2-hydroxyethyl) terephthalate (BHET)), within the range of 240-300 nm is used to detect breakdown of solid PET.

FIG. 5 shows the melting temperature (Tm) of AncG4 (82.4° C.), and AncG4_N205C_S252C_F210V (80.6° C.) compared to extant cutinase E5BBQ3 (TfCut2) (73.9° C.), as determined by differential scanning fluorimetry (DSF).

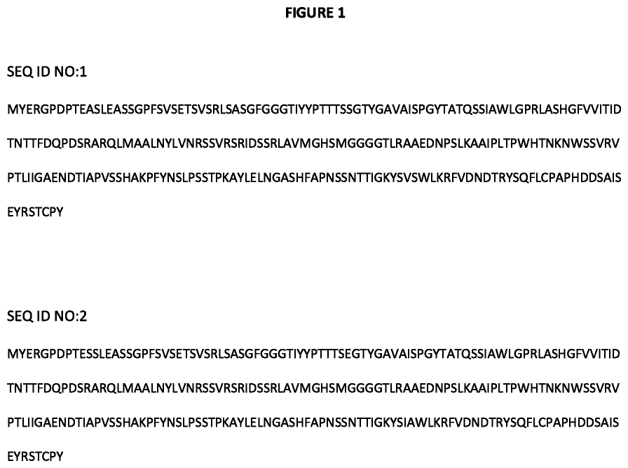

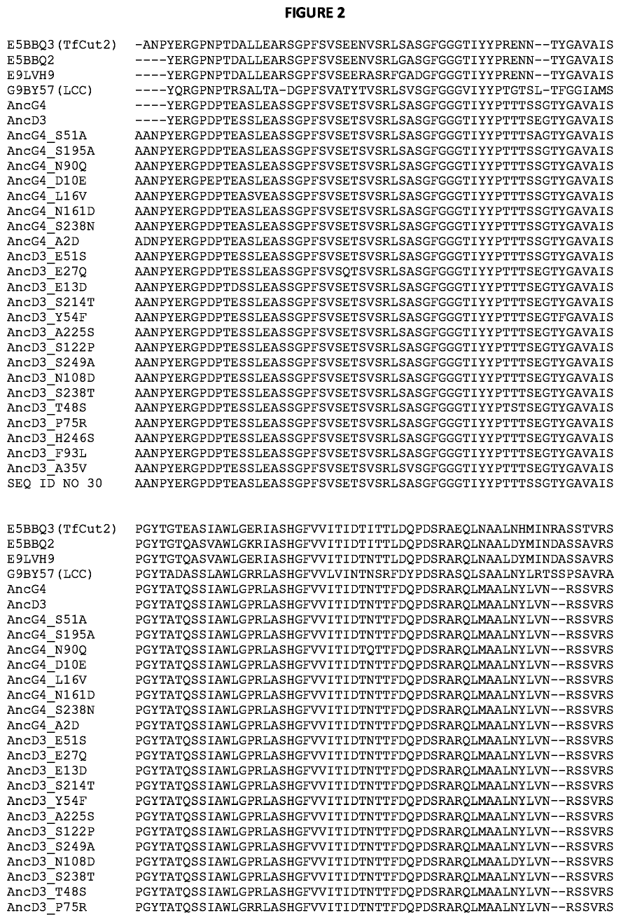

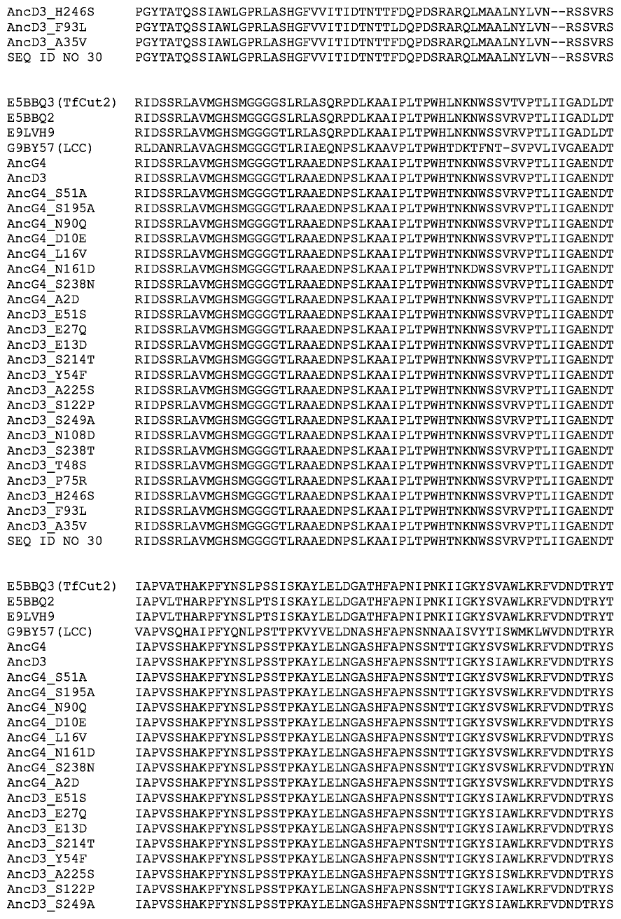

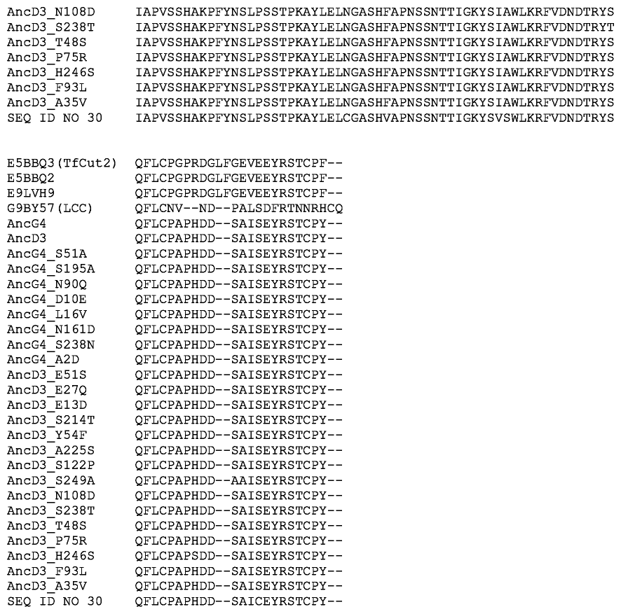

### DETAILED DESCRIPTION OF THE INVENTION

Unless defined otherwise, all technical and scientific terms used herein have the same meaning as commonly understood by those of ordinary skill in the art to which the invention belongs. Although any methods and materials similar or equivalent to those described herein can be used in the practice or testing of the present invention, preferred methods and materials are described. For the purposes of the present invention, the following terms are defined below.

The articles “a” and “an” are used herein to refer to one or to more than one (i.e., to at least one) of the grammatical object of the article, unless explicitly stated otherwise. By way of example, “an element” means one element or more than one element.

As used herein, the term “about” refers to a quantity, level, value, dimension, size, or amount that varies by as much as 10% (e.g, by 10%, 9%, 8%, 7%, 6%, 5%, 4%, 3%, 2% or 1%) to a reference quantity, level, value, dimension, size, or amount.

Throughout this specification, unless the context requires otherwise, the words “comprise”, “comprises” and “comprising” will be understood to imply the inclusion of a stated step or element or group of steps or elements but not the exclusion of any other step or element or group of steps or elements.

This invention relates to the design and production of synthetic esterases with improved hydrolase activity, such as high activity, or broader activity on the mono-, di- and poly-terephthalate esters of PET. The invention is at least partly predicated on the surprising discovery that said engineered polypeptide, from the ancestral sequence reconstruction of extant cutinases may have one or more increased or enhanced properties relative to one or more extant PETases or cutinases, including those having PETase activity. For example, in certain embodiments an engineered ancestral enzyme of the invention has increased thermal stability.

This is a highly surprising discovery because the temperature conditions to which a hypothetical ancestral enzyme may have been exposed would not be too dissimilar to the temperature or conditions to which one or more corresponding extant enzymes are exposed to.

Thus in an aspect disclosed herein, there is provided a polypeptide having esterase activity, wherein the polypeptide comprises an amino acid sequence that has at least 80% identity to amino acids 2-257 of SEQ ID NO:1. There is also provided a polypeptide having esterase activity, wherein the polypeptide comprises an amino acid sequence that has at least 85% identity to amino acids 2-257 of SEQ ID NO:1.

In another embodiment, the polypeptide disclosed herein has improved hydrolase activity, such as high activity, or broader activity on the mono-, di- and poly-terephthalate esters of PET. In yet another embodiment, the polypeptide disclosed herein of the invention has improved hydrolase activity on the mono- and di-terephthalate esters of PET.

The present inventors have also unexpectedly found that substitutions can be made to the engineered, synthetic esterase to advantageously modify or enhance its hydrolase or esterase activity and/or thermostability when compared to extant PETases and cutinases, including those having PETase activity.

By “at least 80%” is meant that the polypeptide shares at least 80%, preferably at least 81%, preferably at least 85%, preferably at least 90%, preferably at least 92%, preferably at least 94%, preferably at least 95%, preferably at least 96%, preferably at least 97%, preferably at least 98%, or more preferably at least 99% sequence identity to SEQ ID NO: 1. As the polypeptide described herein is a synthetic polypeptide, it is to be understood that, in this context, “at least 80%” can include 100% sequence identity across the entire sequence of SEQ ID NO:1. In some embodiments, the polypeptide may comprise amino acid insertions and/or deletions, such as at the N- and/or C-termini, as long as the modified polypeptide has hydrolase activity when compared to the extant cutinases or PETases or the polypeptide having the amino acid sequence of SEQ ID NO:1, as described herein.

In embodiments disclosed herein, there is provided polypeptides that comprise an amino acid sequence that differs from SEQ ID NO:1 by an amino acid substitution at one or more positions corresponding to amino acid position selected from the group consisting of:
xxii. a position that corresponds to amino acid position 7 of SEQ ID NO:1;
xxiii. a position that corresponds to amino acid position 10 of SEQ ID NO:1;
xxiv. a position that corresponds to amino acid position 11 of SEQ ID NO:1;
xxv. a position that corresponds to amino acid position 13 of SEQ ID NO:1;
xxvi. a position that corresponds to amino acid position 24 of SEQ ID NO:1;
xxvii. a position that corresponds to amino acid position 32 of SEQ ID NO:1;
xxviii. a position that corresponds to amino acid position 45 of SEQ ID NO:1;
xxix. a position that corresponds to amino acid position 48 of SEQ ID NO:1;
xxx. a position that corresponds to amino acid position 51 of SEQ ID NO:1;
xxxi. a position that corresponds to amino acid position 72 of SEQ ID NO:1;
xxxii. a position that corresponds to amino acid position 87 of SEQ ID NO:1;
xxxiii. a position that corresponds to amino acid position 90 of SEQ ID NO:1;
xxxiv. a position that corresponds to amino acid position 105 of SEQ ID NO:1;
xxxv. a position that corresponds to amino acid position 119 of SEQ ID NO:1;
xxxvi. a position that corresponds to amino acid position 158 of SEQ ID NO:1;
xxxvii. a position that corresponds to amino acid position 192 of SEQ ID NO:1;
xxxviii. a position that corresponds to amino acid position 211 of SEQ ID NO:1;
xxxix. a position that corresponds to amino acid position 222 of SEQ ID NO:1;
xl. a position that corresponds to amino acid position 235 of SEQ ID NO:1;
xli. a position that corresponds to amino acid position 243 of SEQ ID NO:1; and
xlii. a position that corresponds to amino acid position 246 of SEQ ID NO:1.

In an embodiment, the polypeptide further comprises an amino acid sequence that differs from SEQ ID NO:1 by an amino acid substitution at one or more positions corresponding to amino acid position selected from the group consisting of:
i. a position that corresponds to amino acid position 202 of SEQ ID NO:1;
ii. a position that corresponds to amino acid position 207 of SEQ ID NO:1; and
iii. a position that corresponds to amino acid position 249 of SEQ ID NO:1.

In an embodiment, the amino acid sequence of the polypeptide differs from SEQ ID NO:1 by an amino acid substitution at a position that corresponds to amino acid position D7 of SEQ ID NO:1.

In an embodiment, the amino acid substitution at a position that corresponds to amino acid position D7 of SEQ ID NO:1 is D7E, or a conservative amino acid substitution thereof.

In an embodiment, the amino acid sequence of the polypeptide differs from SEQ ID NO:1 by an amino acid substitution at a position that corresponds to amino acid position L13 of SEQ ID NO:1.

In an embodiment, the amino acid substitution at a position that corresponds to amino acid position L13 of SEQ ID NO:1 is L13V, or a conservative amino acid substitution thereof.

In an embodiment, the amino acid sequence of the polypeptide differs from SEQ ID NO:1 by an amino acid substitution at a position that corresponds to amino acid position S48 of SEQ ID NO:1.

In an embodiment, the amino acid substitution at a position that corresponds to amino acid position S48 of SEQ ID NO:1 is S48A, or a conservative amino acid substitution thereof.

In an embodiment, the amino acid sequence of the polypeptide differs from SEQ ID NO:1 by an amino acid substitution at a position that corresponds to amino acid position N87 of SEQ ID NO:1.

In an embodiment, the amino acid substitution at a position that corresponds to amino acid position N87 of SEQ ID NO:1 is N87Q, or a conservative amino acid substitution thereof.

In an embodiment, the amino acid sequence of the polypeptide differs from SEQ ID NO:1 by an amino acid substitution at a position that corresponds to amino acid position N158 of SEQ ID NO:1.

In an embodiment, the amino acid substitution at a position that corresponds to amino acid position N158 of SEQ ID NO:1 is N158D, or a conservative amino acid substitution thereof.

In an embodiment, the amino acid sequence of the polypeptide differs from SEQ ID NO:1 by an amino acid substitution at a position that corresponds to amino acid position S192 of SEQ ID NO:1.

In an embodiment, the amino acid substitution at a position that corresponds to amino acid position S192 of SEQ ID NO:1 is S192A, or a conservative amino acid substitution thereof.

In an embodiment, the amino acid sequence of the polypeptide differs from SEQ ID NO:1 by an amino acid substitution at a position corresponding to amino acid position N202 of SEQ ID NO:1.

In an embodiment, the amino acid substitution at a position that corresponds to amino acid position N202 of SEQ ID NO:1 is N202C, or a conservative amino acid substitution thereof.

In an embodiment, the amino acid sequence of the polypeptide differs from SEQ ID NO:1 by an amino acid substitution at a position corresponding to amino acid position F207 of SEQ ID NO:1.

In an embodiment, the amino acid substitution at a position that corresponds to amino acid position F207 of SEQ ID NO:1 is F207V, or a conservative amino acid substitution thereof.

In an embodiment, the amino acid sequence of the polypeptide differs from SEQ ID NO:1 by an amino acid substitution at a position that corresponds to amino acid position S235 of SEQ ID NO:1.

In an embodiment, the amino acid substitution at a position that corresponds to amino acid position S235 of SEQ ID NO:1 is S235N, or a conservative amino acid substitution thereof.

In an embodiment, the amino acid sequence of the polypeptide differs from SEQ ID NO:1 by an amino acid substitution at a position corresponding to amino acid position S249 of SEQ ID NO:1.

In an embodiment, the amino acid substitution at a position that corresponds to amino acid position S249 of SEQ ID NO:1 is S249C, or a conservative amino acid substitution thereof.

The present disclosure also contemplates combinations of amino acid substitutions at two or more (e.g., 2, 3, 4, 5, 6, 7, 8, 9, 10 and so on) positions corresponding to positions in SEQ ID NO:1, as described herein. In an embodiment, the polypeptide comprises a combination of amino acid substitutions of at least 2, preferably at least 3, preferably at least 4, preferably at least 5, preferably at least 6, preferably at least 7, preferably at least 8, preferably at least 9, or more preferably at least 10 positions corresponding to positions in SEQ ID NO:1, as described herein.

In an embodiment, the amino acid sequence of the polypeptide differs from SEQ ID NO:1 by amino acid substitutions at positions that corresponds to amino acid positions N202, F207 and S249 of SEQ ID NO:1.

In an embodiment, the amino acid substitutions corresponding to amino acid positions N202, F207 and S249 of SEQ ID NO:1 is N202C, F207V and S249C, or a conservative amino acid substitution thereof.

In an embodiment, the amino acid sequence of the polypeptide differs from SEQ ID NO:1 by amino acid substitutions at positions that corresponds to amino acid positions A11, S48 and V221 of SEQ ID NO:1.

In an embodiment, the amino acid substitutions corresponding to amino acid positions A11, S48 and V221 of SEQ ID NO:1 is A11S, S48E, and V221I, or a conservative amino acid substitution thereof.

In an embodiment, the amino acid sequence of the polypeptide differs from SEQ ID NO:1 by amino acid substitutions at positions that corresponds to amino acid positions A11, S48 and V221 of SEQ ID NO:1.

In an embodiment, the amino acid substitutions position corresponding to amino acid positions A11, S48 and V221 of SEQ ID NO:1 is A11S, S48E, and V221I, or a conservative amino acid substitution thereof.

In an embodiment, the amino acid sequence of the polypeptide differs from SEQ ID NO:1 by amino acid substitutions at positions that corresponds to amino acid positions A11, V221 and S222A of SEQ ID NO:1.

In an embodiment, the amino acid substitutions corresponding to amino acid positions A11, V221 and S222A of SEQ ID NO:1 is A11S, V221I and S222A, or a conservative amino acid substitution thereof.

In an embodiment, the amino acid sequence of the polypeptide differs from SEQ ID NO:1 by amino acid substitutions at positions that corresponds to amino acid positions A11, S48, V221 and S222 of SEQ ID NO:1.

In an embodiment, the amino acid substitutions corresponding to amino acid positions A11, S48, V221 and S222 of SEQ ID NO:1 is A11S, S48E, V221I and S222A, or a conservative amino acid substitution thereof.

In an embodiment, the amino acid sequence of the polypeptide differs from SEQ ID NO:1 by amino acid substitutions at positions that corresponds to amino acid positions E10, A11, S48, V221 and S222 of SEQ ID NO:1.

In an embodiment, the amino acid substitutions corresponding to amino acid positions E10, A11, S48, V221 and S222 of SEQ ID NO:1 is E10D, A11S, S48E, V221I and S222A, or a conservative amino acid substitution thereof.

In an embodiment, the amino acid sequence of the polypeptide differs from SEQ ID NO:1 by amino acid substitutions at positions that corresponds to amino acid positions A11, E24, S48, V221 and S222 of SEQ ID NO:1.

In an embodiment, the amino acid substitutions corresponding to amino acid positions A11, E24, S48, V221 and S222 of SEQ ID NO:1 is A11S, E24Q, S48E, V221I and S222A, or a conservative amino acid substitution thereof.

In an embodiment, the amino acid sequence of the polypeptide differs from SEQ ID NO:1 by amino acid substitutions at positions that corresponds to amino acid positions A11, A32, S48, V221 and S222 of SEQ ID NO:1.

In an embodiment, the amino acid substitutions corresponding to amino acid positions A11, A32, S48, V221 and S222 of SEQ ID NO:1 is A11S, A32V, S48E, V221I and S222A, or a conservative amino acid substitution thereof.

In an embodiment, the amino acid sequence of the polypeptide differs from SEQ ID NO:1 by amino acid substitutions at positions that corresponds to amino acid positions A11, T45, S48, V221 and S222 of SEQ ID NO:1.

In an embodiment, the amino acid substitutions positions that corresponds to amino acid positions A11, T45, S48, V221 and S222 of SEQ ID NO:1 is A11S, T45S, S48E, V221I and S222A, or a conservative amino acid substitution thereof.

In an embodiment, the amino acid sequence of the polypeptide differs from SEQ ID NO:1 by amino acid substitutions at positions that corresponds to amino acid positions A11, S48, Y51, V221 and S222 of SEQ ID NO:1.

In an embodiment, the amino acid substitutions corresponding to amino acid positions A11, S48, Y51, V221 and S222 of SEQ ID NO:1 is A11S, S48E, Y51F, V221I and S222A, or a conservative amino acid substitution thereof.

In an embodiment, the amino acid sequence of the polypeptide differs from SEQ ID NO:1 by amino acid substitutions at positions that corresponds to amino acid positions A11, S48, P72, V221 and S222 of SEQ ID NO:1.

In an embodiment, the amino acid substitutions corresponding to amino acid positions A11, S48, P72, V221 and S222 of SEQ ID NO:1 is A11S, S48E, P72R, V221I and S222A, or a conservative amino acid substitution thereof.

In an embodiment, the amino acid sequence of the polypeptide differs from SEQ ID NO:1 by amino acid substitutions at positions that corresponds to amino acid positions A11, S48, F90, V221 and S222 of SEQ ID NO:1.

In an embodiment, the amino acid substitutions corresponding to amino acid positions A11, S48, F90, V221 and S222 of SEQ ID NO:1 is A11S, S48E, F90L, V221I and S222A, or a conservative amino acid substitution thereof.

In an embodiment, the amino acid sequence of the polypeptide differs from SEQ ID NO:1 by amino acid substitutions at positions that corresponds to amino acid positions A11, S48, N105, V221 and S222 of SEQ ID NO:1.

In an embodiment, the amino acid substitutions corresponding to amino acid positions A11, S48, N105, V221 and S222 of SEQ ID NO:1 is A11S, S48E, N105D, V221I and S222A, or a conservative amino acid substitution thereof.

In an embodiment, the amino acid sequence of the polypeptide differs from SEQ ID NO:1 by amino acid substitutions at positions that corresponds to amino acid positions A11, S48, S119, V221 and S222 of SEQ ID NO:1.

In an embodiment, the amino acid substitutions corresponding to amino acid positions A11, S48, S119, V221 and S222 of SEQ ID NO:1 is A11S, S48E, S119P, V221I and S222A, or a conservative amino acid substitution thereof.

In an embodiment, the amino acid sequence of the polypeptide differs from SEQ ID NO:1 by amino acid substitutions at positions that corresponds to amino acid positions A11, S48, S211, V221 and S222 of SEQ ID NO:1.

In an embodiment, the amino acid substitutions corresponding to amino acid positions A11, S48, S211, V221 and S222 of SEQ ID NO:1 is A11S, S48E, S211T, V221I and S222A, or a conservative amino acid substitution thereof.

In an embodiment, the amino acid sequence of the polypeptide differs from SEQ ID NO:1 by amino acid substitutions at positions that corresponds to amino acid positions A11, S48, V221, S222 and S235 of SEQ ID NO:1.

In an embodiment, the amino acid substitutions corresponding to amino acid positions A11, S48, V221, S222 and S235 of SEQ ID NO:1 is A11S, S48E, V221I, S222A and S235T or a conservative amino acid substitution thereof.

In an embodiment, the amino acid sequence of the polypeptide differs from SEQ ID NO:1 by amino acid substitutions at positions that corresponds to amino acid positions A11, S48, V221, S222 and H243 of SEQ ID NO:1.

In an embodiment, the amino acid substitutions corresponding to amino acid positions A11, S48, V221, S222 and H243 of SEQ ID NO:1 is A11S, S48E, V221I, S222A and H243S or a conservative amino acid substitution thereof.

In an embodiment, the amino acid sequence of the polypeptide differs from SEQ ID NO:1 by amino acid substitutions at positions that corresponds to amino acid positions A11, S48, V221, S222 and S246 of SEQ ID NO:1.

In an embodiment, the amino acid substitution corresponding to amino acid positions A11, S48, V221, S222 and S246 of SEQ ID NO:1 is A11S, S48E, V221I, S222A and S246A or a conservative amino acid substitution thereof.

In another embodiment, the polypeptide comprises an N-terminal cellular export signal peptide. In an embodiment, the N-terminal cellular export signal peptide comprises the amino acid sequence of SEQ ID NO:28 or SEQ ID NO:29. In a preferred embodiment, the N-terminal cellular export signal peptide comprises the amino acid sequence of SEQ ID NO:28.

The present disclosure extends to polypeptides that comprise an amino acid sequence selected from the group consisting SEQ ID NOs:2-27 and 30, or an amino acid sequence having at least 80% sequence identity thereto.

The present disclosure also extends to a composition comprising the polypeptide as described herein.

The present disclosure also extends to a nucleic acid sequence encoding the polypeptide described herein.

The present disclosure also extends to an expression vector comprising the nucleic acid sequence described herein.

The present disclosure also extends to a host cell comprising the nucleic acid sequence or the expression vector described herein.

In another aspect, the present disclosure provides a method of producing a polypeptide having esterase activity, the method comprising:
i. providing the polynucleotide described herein;
ii. expressing the polynucleotide in a host cell under conditions sufficient to allow the host cell to produce the polypeptide; and
iii. collecting the polypeptide produced by the host cell in ii.

In yet another aspect, there is provided a method for hydrolysing a terephthalic acid ester, the method comprising contacting a terephthalic acid ester with the polypeptide, the composition, or the host cell described herein, under conditions sufficient to convert the terephthalic acid ester to terephthalic acid and ethylene glycol, mono-(2-hydroxyethyl) terephthalate and/or bis-(2-hydroxyethyl) terephthalate.

The present disclosure also extends to a method of degrading a plastic product comprising a polyester, the method comprising contacting the plastic product with the polypeptide, the composition, or the host cell described herein, under conditions sufficient to degrade the plastic product.

In an embodiment, the polyester is selected from the group consisting of polylactic acid (PLA), polytrimethylene terephthalate (PTT), polybutylene terephthalate (PBT), polyethylene isosorbide terephthalate (PEIT), polyethylene terephthalate (PET) polyhydroxyalkanoate (PHA), polybutylene succinate (PBS), polybutylene succinate adipate (PBSA), polybutylene adipate terephthalate (PBAT), polyethylene furanoate (PEF), polycapro lactone (PCL), polyethylene adipate (PEA), poly(glycolic acid) (PGA), poly(lactic-co-glycolic acid) (PLGA) and combinations of any of the foregoing. In an embodiment the polyester is polyethylene terephthalate (PET).

In another embodiment, the methods disclosed herein comprise:
i. exposing the PET to the polypeptides, compositions or host cells disclosed herein under conditions sufficient for the polypeptides (which may be contained in the compositions or expressed by the host cells) to catalyze the conversion of the PET to generate terephthalic acid, mono-(2-hydroxyethyl) terephthalate and/or bis-(2-hydroxyethyl) terephthalate; and
ii. simultaneously or sequentially, exposing the PET to
   a. a mono-(2-hydroxyethyl) terephthalate hydrolase (MHETase), or an enzyme having MHETase activity;
   b. a wild-type or extant PETase; and/or
   c. a cutinase having PETase activity.

The present disclosure also extends to a composition comprising the terephthalic acid and/or ethylene glycol recovered by the method disclosed herein.

In another aspect, there is provided a host cell genetically modified to express the polypeptide described herein.

In another aspect, there is provided a method of producing a plastic product using the composition of the terephthalic acid and ethylene glycol generated by the method disclosed herein.

The term “wild-type” is used herein to denote a naturally-occurring isoform of a polypeptide; that is, as it appears in nature. The term “extant” is used to denote naturally-occurring isoform of a polypeptide found in organisms or species still in existence (i.e. not extinct).

Examples of extant cutinases would be familiar to the persons skilled in the art.

Examples of extant cutinases include, but are not limited to: Leaf-branch compost cutinase (LCC; Accession number: G9BY57), TfCut2 (cutinase from Thermobifida fusca; Accession number: E5BBQ3), E9LVH9 (Cut2 from Thermobifida cellulosilytica) and E5BBQ2 (Cut.1-KW3 cutinase from Thermobifida fusca).

Herein, the terms “peptide”, “polypeptide”, “protein”, “enzyme” are to be understood as referring to a chain of amino acids linked by peptide bonds, irrespective of the number of amino acids forming said chain. The amino acids are typically represented by their one-letter or three-letters code, according to the following nomenclature: A: alanine (Ala); C: cysteine (Cys); D: aspartic acid (Asp); E: glutamic acid (Glu); F: phenylalanine (Phe); G: glycine (Gly); H: histidine (His); I: isoleucine (Ile); K: lysine (Lys); L: leucine (Leu); M: methionine (Met); N: asparagine (Asn); P: proline (Pro); Q: glutamine (Gln); R: arginine (Arg); S: serine (Ser); T: threonine (Thr); V: valine (Val); W: tryptophan (Trp) and Y: tyrosine (Tyr).

The term “hydrolase” refers to an enzyme which belongs to a class of hydrolases classified as EC 3 according to Enzyme Nomenclature that catalyzes the hydrolysis of chemical bonds, including ester bonds.

The term “esterase” refers to a hydrolase enzyme which is classified as EC 3.1 according to Enzyme Nomenclature that catalyzes the hydrolysis of ester bonds to produce an acid and an alcohol. The term “cutinase” refers to a serine esterase enzyme which is classified as EC 3.1.1.74 according to Enzyme Nomenclature, which catalyses the hydrolysis of cutin polymers (a polyester composed of hydroxy and hydroxyepoxy fatty acids) into cutin monomers.

The term “PETase” refers to an esterase enzyme, which is classified as EC 3.1.1.101 according to Enzyme Nomenclature that catalyzes the hydrolysis of polyethylene terephthalate (PET) plastic to monomeric mono-2-hydroxyethyl terephthalate (MHET).

Herein, the polypeptide of the present invention is understood to have hydrolase or esterase activity, having capability to catalyse the hydrolysis of mono- and di-, and poly-terephthalic acid esters.

As noted by Palm et al. (2019, Nat. Comms. 10:1717), two recently discovered bacterial enzymes that specifically degrade polyethylene terephthalate (PET) represent a promising solution to an otherwise environmentally burdensome polyester containing product. First, Ideonella sakaiensis PETase, a structurally well-characterized α/β-hydrolase fold enzyme, converts PET to mono-(2-hydroxyethyl) terephthalate (MHET). MHETase, the second key enzyme, hydrolyzes MHET to terephthalate and ethylene glycol (Palm et al. (2019, Nat. Comm., 10:1717), Sagong et al. (2020, ACS Catal. 10:4805) and Yoshida et al. (2016, Science, 352(6278):1196).

The terms “mutant” and “variant” may be used interchangeably herein to refer to a polypeptide comprising an amino acid sequence that is derived from SEQ ID NO:1 and further comprising a modification or alteration (e.g., a substitution, insertion, and/or deletion), at one or more (e.g., several) positions and having enhanced esterase activity in catalysing the hydrolysis of mono- and di-, and poly-terephthalic acid esters, when compared to extant PETases or cutinases, including those having PETase activity or the polypeptide of SEQ ID NO:1. Such variants may be obtained by various techniques well known in the art, illustrative examples of which include site-directed mutagenesis, random mutagenesis and synthetic oligonucleotide construction. The terms “modification”, “alteration”, “substitution” and the like, as used herein in relation to an amino acid residue or position, typically mean that the amino acid in the particular position has been modified compared to the amino acid of the wild-type or parent polypeptide.

Suitable substitutions may include the replacement of an amino acid residue by another selected from the naturally-occurring standard 20 amino acid residues, rare naturally occurring amino acid residues (e.g., hydroxyproline, hydroxylysine, allohydroxylysine, 6-N-methylysine, N-ethylglycine, N-methylglycine, N-ethylasparagine, allo-isoleucine, N-methylisoleucine, N-methylvaline, pyroglutamine, aminobutyric acid, ornithine, norleucine, norvaline), and non-naturally occurring amino acid residue, often made synthetically, (e.g., cyclohexyl-alanine). Preferably, the substitution comprises the replacement of an amino acid residue by another selected from the naturally-occurring standard 20 amino acid residues (G, P, A, V, L, I, M, C, F, Y, W, H, K, R, Q, N, E, D, S and T). The modification or alteration may be identified herein using the following terminology: Y197V denotes that amino acid residue Tyrosine (Y) at position 197 of the parent polypeptide sequence is substituted for a Valine (V). Y197V/I/M denotes that amino acid residue Tyrosine (Y) at position 197 of the parent sequence may be substituted for one of the following amino acids: Valine (V), Isoleucine (I), or Methionine (M). The substitution can be a conservative or non-conservative substitution. Examples of conservative substitutions will be familiar to persons skilled in the art, illustrative examples of which include substitutions within the groups of basic amino acids (arginine, lysine and histidine), acidic amino acids (glutamic acid and aspartic acid), polar amino acids (glutamine, asparagine and threonine), hydrophobic amino acids (methionine, leucine, isoleucine, cysteine and valine), aromatic amino acids (phenylalanine, tryptophan and tyrosine), and small amino acids (glycine, alanine and serine).

Unless otherwise specified, the positions disclosed in the present application are numbered by reference to the amino acid sequence set forth in SEQ ID NO:1. In this context, the term “corresponding to”, when used in reference to an amino acid position, is intended to mean an amino acid position in a polypeptide sequence when that position is aligned with the equivalent or corresponding position in the sequence set forth in SEQ ID NO:1.

As used herein, the term “sequence identity” or “identity” refers to the number (or fraction expressed as a percentage %) of matches (identical amino acid residues) between two polypeptide sequences. In a preferred embodiment, the sequence identity is determined by comparing the sequences when aligned so as to maximize overlap and identity while minimizing sequence gaps. Sequence identity may be determined using any of a number of mathematical global or local alignment algorithms known to persons skilled in the art, depending on the length of the two sequences. Sequences of similar lengths may be aligned using a global alignment algorithms (e.g., Needleman and Wunsch algorithm; Needleman and Wunsch, 1970), which aligns the sequences optimally over the entire length, while sequences of substantially different lengths are preferably aligned using a local alignment algorithm (e.g., Smith and Waterman algorithm (Smith and Waterman, 1981) or Altschul algorithm (Altschul et al., 1997; Altschul et al., 2005)). Alignment for the purposes of determining percent amino acid sequence identity can be achieved by any means available to persons skilled in the art, illustrative examples of which include publicly available computer software, such as is available at http://blast.ncbj.nlm.nih.gov/ or https://www.ebi.ac.uk//Tools/emboss/). Persons skilled in the art can readily determine appropriate parameters for measuring alignment, including any algorithms needed to achieve maximal alignment over the full length of the sequences being compared. As used herein, % sequence identity typically refers to values generated using pair wise sequence alignment that creates an optimal global alignment of two sequences (e.g., using the Needleman-Wunsch algorithm), where all search parameters are set to default values, e.g., Scoring matrix=BLOSUM62, Gap open=10, Gap extend=0.5, End gap penalty=false, End gap open=10 and End gap extend=0.5.

The term “recombinant”, as used herein, typically refers to a nucleic acid construct, a vector, a polypeptide or a cell produced by genetic engineering.

The term “expression”, as used herein, typically refers to any step involved in the production of a polypeptide, such as by transcription, post-transcriptional modification, translation, post-translational modification, and secretion.

The term “expression cassette” denotes a nucleic acid construct comprising a coding region, and suitably a regulatory region to which the coding region is operably linked.

The term “expression vector” typically means a DNA or RNA molecule that comprises an expression cassette. The expression vector may be a linear or circular double stranded DNA molecule.

The term “polymer”, as used herein, typically refers to a chemical compound or a mixture of compounds whose structure is made up of multiple monomers (repeat units) linked by covalent chemical bonds. Within the context of the invention, the term polymer includes natural or synthetic polymers, constituted of a single type of repeat unit (i.e., homopolymers) or of a mixture of different repeat units (i.e., copolymers or heteropolymers).

As used herein, the terms “polyester containing material”, “polyester containing product” and the like are to be understood as refers to a product, such as plastic product, comprising at least one polyester in crystalline, semi-crystalline or totally amorphous form. The polyester containing material may refer to any item made from at least one plastic material, such as plastic sheet, tube, rod, profile, shape, film, massive block, fiber, textiles, etc., which contains at least one polyester, and possibly other substances or additives, such as plasticizers, mineral or organic fillers. In an embodiment, the polyester containing material is a textile or fabric comprising at least one polyester containing fiber. In another embodiment, the polyester containing material is a plastic compound, or plastic formulation, in a molten or solid state, suitable for making a plastic product.

Suitable polyesters will be familiar to persons skilled in the art, illustrative examples of which include polylactic acid (PLA), polyethylene terephthalate (PET), polytrimethylene terephthalate (PTT), polybutylene terephthalate (PBT), polyethylene isosorbide terephthalate (PEIT), polyhydroxyalkanoate (PHA), polybutylene succinate (PBS), polybutylene succinate adipate (PBSA), polybutylene adipate terephthalate (PBAT), polyethylene furanoate (PEF), polycaprolactone (PCL), and poly(ethylene adipate) (PEA). Thus, in an embodiment, the polyester is selected from the group consisting of polylactic acid (PLA), polyethylene terephthalate (PET), polytrimethylene terephthalate (PTT), polybutylene terephthalate (PBT), polyethylene isosorbide terephthalate (PEIT), polyhydroxyalkanoate (PHA), polybutylene succinate (PBS), polybutylene succinate adipate (PBSA), polybutylene adipate terephthalate (PBAT), polyethylene furanoate (PEF), polycaprolactone (PCL), poly(ethylene adipate) (PEA) and combinations of any of the foregoing.

As noted elsewhere herein, the present inventors have developed novel polypeptides having esterase activity. The polypeptides suitably comprise an amino acid sequence that has at least 80% identity to SEQ ID NO: 1. Typically, the polypeptides exhibit increased thermal stability and/or improved esterase activity on the terephthalate esters of PET, including mono- and di-, and poly-terephthalic acid esters, when compared to extant PETases or extant cutinases, including those having PETase activity. This improved enzyme activity can be increased catalytic activity and/or having a broader catalytic activity. More particularly, the inventors have developed novel esterases having improved enzyme activity on the mono- and di-benzyl terephthalate esters of PET for use in industrial processes.

With the aim of improving the activity of esterases, in particular esterases for use in environmental degradation of plastic products, the inventors have performed ancestral sequence reconstruction (ASR) to engineer novel polypeptides comprising an amino acid sequence of SEQ ID NO:1 or amino acid sequences having at least 80% sequence identity thereto, including modifications to SEQ ID NO:1 that unexpectedly result in higher and/or broader catalytic activity when compared to extant PETases or extant cutinases having PETase activity. The engineered enzymes disclosed herein are particularly suited for the degradation of plastic products, in particular those containing PET. Moreover, the inventors have surprisingly found that amino acid residues that are not otherwise intended to contact a polyester substrate in the structure of the protein may be advantageously modified to enhance the hydrolase activity of the variant esterase.

Thus, in another aspect disclosed herein, there is provided a polypeptide capable of catalysing the hydrolysis of mono- and di-, and poly-terephthalic acid esters, with superior hydrolase activity against mono- and di-terephthalic acid esters, wherein the polypeptide comprises an amino acid sequence that (i) has at least 80% sequence identity to SEQ ID NO:1 and (ii) differs from SEQ ID NO:1 by an amino acid substitution at one or more amino acid positions.

In an embodiment, the polypeptide comprises an amino acid sequence that differs from SEQ ID NO:1 by an amino acid substitution at one or more positions that do not make contact with a polyester substrate. The term “contact”, in this context, typically refers to direct contact made by amino acid residues of the polypeptide of SEQ ID NO:1 and variants disclosed herein, with a polyester substrate thereof. In an embodiment, the polypeptide comprises an amino acid sequence that differs from SEQ ID NO:1 by an amino acid substitution at one position that is outside of the catalytic “active site” of the esterase of SEQ ID NO:1. In an embodiment, the polypeptide comprises an amino acid sequence that differs from SEQ ID NO:1 by an amino acid substitution at more than one position outside of the catalytic “active site” of the esterase of SEQ ID NO:1. The term “active site” typically refers to the region of SEQ ID NO:1 that is capable of making contact with and hydrolyzing the polyester substrate (i.e., mono- and di-, and poly-terephthalic acid esters).

In the context of the present disclosure, reference to increased or enhanced hydrolase/esterase activity indicates an increased ability of the polypeptides/novel engineered polypeptides and its variants to hydrolyze mono- and di-, and poly-terephthalic acid esters, when compared to extant cutinases or extant PETases, including those having PETase activity. In an embodiment, the activity of the polypeptide described herein is increased by at least about 5%, preferably by at least about 10%, preferably by at least about 20%, preferably by at least about 30%, preferably by at least about 40%, preferably by at least about 50%, preferably by at least about 100%, preferably by at least about 200%, preferably by at least about 300%, preferably by at least about 400%, preferably by at least about 500%, preferably by at least about 600%, preferably by at least about 700%, preferably by at least about 800%, preferably by at least about 900%, or more preferably by at least about 1,000% or more in comparison to extant PETases or cutinases, including those having PETase activity.

Suitable methods of determining or measuring the esterase/hydrolase activity of a polypeptide will be familiar to persons skilled in the art, an illustrative example of which is described elsewhere herein. Other illustrative examples are described in Palm et al. (2019, Nat. Comm., 10:1717), Sagong et al. (2020, ACS Catal. 10:4805) and Yoshida et al. (2016, Science, 352(6278):1196), the contents of which are incorporated herein by reference in their entirety. In an embodiment, the hydrolase activity is increased by at least about 5%, preferably by at least about 10%, preferably by at least about 20%, preferably by at least about 30%, preferably by at least about 40%, preferably by at least about 50%, preferably by at least about 100%, preferably by at least about 200%, preferably by at least about 300%, preferably by at least about 400%, preferably by at least about 500%, preferably by at least about 600%, preferably by at least about 700%, preferably by at least about 800%, preferably by at least about 900%, or more preferably by at least about 1,000% or more in comparison to extant PETases or cutinases, including those having PETase activity when determined by UV absorbance assay to monitor the amount of product or terephthalic acid produced from using amorphous PET as a substrate. Another method useful for determining or measuring the esterase/hydrolase activity of a polypeptide is by measuring the amount of terephthalic acid produced using Analytical High Performance Liquid Chromatography (HPLC).

The hydrolase activity of the novel engineered polypeptide and variants having esterase activity may be assigned an absolute value or a value relative to the esterase or hydrolase activity of a comparator (e.g., extant PETases or cutinases having PETase activity). In an embodiment, the hydrolase activity is measured as the rate of monomers and/or oligomers (e.g., in mg or mol) released per hour and per mg or mol of enzyme under suitable conditions of temperature, pH and buffer. In an embodiment of the invention, the rate of monomers and/or oligomers released per hour is in the range of from about 1 mol/h/mol enzyme to about 500 mol/h/mol enzyme. In another embodiment, the rate of monomers and/or oligomers (e.g., in mg) released per hour is in the range of from about 50 mol/h/mol enzyme to about 400 mol/h/mol enzyme. In yet another embodiment, the rate of monomers and/or oligomers (e.g., in mg) released per hour is in the range of from about 100 mol/h/mol enzyme to about 350 mol/h/mol enzyme.

Advantageously, the polypeptide described herein may be able to catalyse the hydrolysis of mono- and di-, and poly-terephthalic acid esters at least in a range of temperatures from about 10° C. to about 80° C., preferably from about 20° C. to about 80° C., preferably from about 30° C. to about 80° C., preferably from about 40° C. to about 80° C., preferably from about 50° C. to about 80° C., even preferably from about 60° C. to about 80° C., even more preferably at about 60° C. to about 70° C., even more preferably at about 60° C. In an embodiment, the polypeptide described herein exhibits hydrolase activity at a temperature from about 10° C. to about 80° C., preferably from about 20° C. to about 80° C., preferably from about 30° C. to about 80° C., preferably from about 40° C. to about 80° C., preferably from about 50° C. to about 80° C., even preferably from about 60° C. to about 80° C., even more preferably at about 60° C. to about 70° C., even more preferably at about 60° C. In an embodiment, the hydrolase activity is measurable at a temperature from about 10° C. to about 70° C., preferably from about 20° C. to about 80° C., preferably from about 30° C. to about 80° C., preferably from about 40° C. to about 80° C., preferably from about 50° C. to about 80° C., even preferably from about 60° C. to about 80° C., even more preferably from about 60° C. to about 70° C., even more preferably at about 60° C. In another particular embodiment, the polyester degrading or depolymerisation activity is still measurable at a temperature from about 10° C. to about 30° C., preferably from about 15° C. to about 28° C., corresponding to the mean temperature in the natural environment (ambient temperature).

In an embodiment, the polypeptide having esterase activity comprises hydrolase activity or catalyses the hydrolysis of mono- and di-, and poly-terephthalic acid esters at a temperature from about 10° C. to about 80° C., preferably from about 20° C. to about 80° C., preferably from about 30° C. to about 80° C., preferably from about 40° C. to about 80° C., preferably from about 50° C. to about 80° C., even preferably from about 60° C. to about 80° C., even more preferably from about 60° C. to about 70° C., even more preferably at about 60° C., of at least about 5%, preferably by at least about 10%, preferably by at least about 20%, preferably by at least about 30%, preferably by at least about 40%, preferably by at least about 50%, preferably by at least about 100%, preferably by at least about 200%, preferably by at least about 300%, preferably by at least about 400%, preferably by at least about 500%, preferably by at least about 600%, preferably by at least about 700%, preferably by at least about 800%, preferably by at least about 900%, or more preferably by at least about 1,000% or more in comparison to the hydrolase activity of extant PETases at the same temperature.

In another particular embodiment, the polypeptide having esterase activity is to catalyse the hydrolysis of mono- and di-, and poly-terephthalic acid esters at a temperature from about 10° C. to about 80° C., preferably from about 20° C. to about 80° C., preferably from about 30° C. to about 80° C., preferably from about 40° C. to about 80° C., preferably from about 50° C. to about 80° C., even preferably from about 60° C. to about 80° C., even more preferably at about 60° C. to about 70° C., even more preferably at about 60° C., of at least about 5%, preferably by at least about 10%, preferably by at least about 20%, preferably by at least about 30%, preferably by at least about 40%, preferably by at least about 50%, preferably by at least about 100%, preferably by at least about 200%, preferably by at least about 300%, preferably by at least about 400%, preferably by at least about 500%, preferably by at least about 600%, preferably by at least about 700%, preferably by at least about 800%, preferably by at least about 900%, or more preferably by at least about 1,000% or more in comparison to the hydrolase activity of extant PETases or cutinases having PETase activity at the same temperature.

In another embodiment, the polypeptide described herein has increased hydrolase/esterase activity against mono- di- and poly-terephthalic acid esters, compared to the extant PETases or cutinases having PETase activity, at a temperature from about 10° C. to about 80° C., at a temperature from about 15° C. to about 70° C., preferably from about 25° C. to about 50° C., even more preferably from about 60° C. to about 70° C. In an embodiment, the polypeptide described herein has hydrolase/esterase activity at a temperature from about 40° C. to about 80° C., preferably from about 50° C. to about 80° C., even preferably from about 60° C. to about 80° C., even more preferably at about 60° C. to about 70° C., even more preferably at about 60° C., of at least about 5%, preferably by at least about 10%, preferably by at least about 20%, preferably by at least about 30%, preferably by at least about 40%, preferably by at least about 50%, preferably by at least about 100%, preferably by at least about 200%, preferably by at least about 300%, preferably by at least about 400%, preferably by at least about 500%, preferably by at least about 600%, preferably by at least about 700%, preferably by at least about 800%, preferably by at least about 900%, or more preferably by at least about 1,000% or more in comparison to the hydrolase/esterase activity of extant PETase or cutinases having PETase activity at the same temperature.

In another particular embodiment, the polypeptides described herein has increased hydrolase/esterase activity, against mono- di- and poly-terephthalic acid esters, when compared to extant PETases or cutinases having PETase activity, at a temperature from about 10° C. to about 80° C., preferably from about 20° C. to about 80° C., preferably from about 30° C. to about 80° C., preferably from about 40° C. to about 80° C., preferably from about 50° C. to about 80° C., even preferably from about 60° C. to about 80° C., even more preferably at about 60° C. to about 70° C., even more preferably at about 60° C. In an embodiment, the polypeptide described herein has hydrolase/esterase activity at between about 10° C. to about 80° C., of at least about 5%, preferably by at least about 10%, preferably by at least about 20%, preferably by at least about 30%, preferably by at least about 40%, preferably by at least about 50%, preferably by at least about 100%, preferably by at least about 200%, preferably by at least about 300%, preferably by at least about 400%, preferably by at least about 500%, preferably by at least about 600%, preferably by at least about 700%, preferably by at least about 800%, preferably by at least about 900%, or more preferably by at least about 1,000% or more in comparison to the hydrolase/esterase activity of extant PETases or cutinases having PETase activity at the same temperature.

In an embodiment, the polypeptide described herein exhibits a measurable hydrolase/esterase activity at least in a range of pH from about 5 to about 11, preferably in a range of pH from about 6 to about 10, preferably in a range of pH from about 7 to about 10, more preferably in a range of pH from about 7.5 to about 9.5.

Advantageously, the thermostability of the polypeptides described herein is not significantly impaired compared to extant PETases or cutinases, including those having PETase activity. In some embodiments, the thermostability of the polypeptide described herein is improved when compared to the thermostability of extant PETases or cutinases, including those having PETase activity. The term “improved thermostability”, as used herein, indicates an increased ability of the enzyme to resist changes in its chemical and/or physical structure at higher temperatures, more specifically at temperature between 40° C. and 80° C., as compared to extant PETases or cutinases, including those having PETase activity. In an embodiment, the polypeptides described herein have an increased half-life at a temperature between 40° C. and 80° C., as compared to extant PETases or cutinases, including those having PETase activity. The polypeptides described herein may exhibit a higher or equivalent melting temperature (Tm) as compared to extant PETases or cutinases, including those having PETase activity. In some embodiments, the polypeptide described herein shows improved thermostability at a temperature of from about 40° C. to about 80° C. as compared to extant PETases or cutinases, including those having PETase activity.

The thermostability of a polypeptide may be evaluated by any suitable means known to persons skilled in the art. For example, thermostability can be assessed by measuring the residual esterase or hydrolase activity of the polypeptide after incubation at different temperatures. The ability to perform multiple rounds of hydrolysis at different temperatures can also be evaluated. Differential Scanning Fluorimetry (DSF) may also be used to assess the thermostability of the polypeptide. Circular dichroism may also be used to measure thermostability of the polypeptides described herein, including their melting temperatures (Tm). The term “melting temperature (Tm)” is understood to mean a given protein corresponds to the temperature at which 50% of said protein is denatured.

In an embodiment, the polypeptide described herein exhibits a melting temperature (Tm) of from about 40° C. to about 90° C., from 50° C. to about 90° C., preferably from about 60° C. to about 90° C., preferably from about 70° C. to about 90° C. In an embodiment, the polypeptide described herein exhibits a melting temperature (Tm) that is higher than the Tm exhibited by extant PETases or cutinases, including those having PETase activity.

As used herein, the term “nucleic acid”, “nucleic sequence” “polynucleotide”, “oligonucleotide” and “nucleotide sequence” are used interchangeably and refer to a sequence of deoxyribonucleotides and/or ribonucleotides. The nucleic acids can be DNA (cDNA or gDNA), RNA, or a mixture of the two. It can be in single stranded form or in duplex form or a mixture of the two. It can be of recombinant, artificial and/or synthetic origin and it can comprise modified nucleotides, comprising for example a modified bond, a modified purine or pyrimidine base, or a modified sugar. The nucleic acids of the invention can be in isolated or purified form, and made, isolated and/or manipulated by techniques known per se in the art, e.g., cloning and expression of cDNA libraries, amplification, enzymatic synthesis or recombinant technology. The nucleic acids can also be synthesized in vitro by well-known chemical synthesis techniques, as described in, e.g., Belousov (1997) Nucleic Acids Res. 25:3440-3444.

The nucleic acid sequences disclosed herein may suitably be codon optimized. Suitable methods for codon optimization will be familiar to persons skilled in the art, illustrative examples of which are described in the reference manual Sambrook et al. (Sambrook et al., 2001).

The nucleic acid sequences described herein may be suitably deduced from the amino acid sequence of the polypeptides described herein and codon usage may be adapted according to the host cell in which the nucleic acid shall be transcribed.

In some embodiments, the nucleic acid sequences described herein may suitably comprise additional nucleotide sequences, such as regulatory regions, i.e., promoters, enhancers, silencers, terminators, signal peptides and the like that can be used to cause or regulate expression of the polypeptide in a selected host cell or system. Alternatively, or in addition, the nucleic acid sequences described herein may further comprise additional nucleotide sequences encoding fusion proteins, such as maltose binding protein (MBP) or glutathion S transferase (GST) that can be used to favor polypeptide expression and/or solubility.

As noted elsewhere herein, the present disclosure also extends to expression vectors and expression cassettes comprising the nucleic acid sequence described herein, optionally operably linked to one or more control sequences that direct the expression of the nucleic acid sequence in a suitable host cell. Typically, the expression vector or cassette comprises the nucleic acid sequence described herein operably linked to a control sequence such as transcriptional promoter and/or transcription terminator. The control sequence may include a promoter that is recognized by a host cell or an in vitro expression system for expression of the nucleic acid encoding the polypeptide described herein. The promoter will typically comprise a transcriptional control sequence that mediates the expression of the polypeptide. The promoter may be any polynucleotide that shows transcriptional activity in a host cell, including mutant, truncated, and hybrid promoters, and may suitably be obtained from genes encoding extracellular or intracellular polypeptides either homologous or heterologous to the host cell. The control sequence may also be a transcription terminator, which is recognized by a host cell to terminate transcription. The terminator is typically operably linked to the 3′-terminus of the nucleic acid encoding the polypeptide. Any terminator that is functional in the host cell may be used in this context. Typically, the expression vector or cassette comprises the nucleic acid sequence described herein operably linked to a transcriptional promoter and a transcription terminator.

The term “vector” typically refers to a DNA molecule used as a vehicle to transfer recombinant genetic material into a host cell. Suitable vectors include plasmids, bacteriophages, viruses, fosmids, cosmids, and artificial chromosomes. The vector is typically a DNA sequence that comprises an insert (a heterologous nucleic acid sequence, transgene) and a larger sequence that serves as the “backbone” of the vector. The purpose of a vector which transfers genetic information to the host is typically to isolate, multiply, or express the insert in the target cell. Expression vectors (also referred to as expression constructs) are specifically adapted for the expression of the heterologous sequences in the target cell, and generally have a promoter sequence that drives expression of the heterologous sequences encoding a polypeptide.

Generally, the regulatory elements that are used in an expression vector include a transcriptional promoter, a ribosome binding site, a terminator, and optionally present operator. An expression vector may further comprise an origin of replication for autonomous replication in a host cell, a selectable marker, a limited number of useful restriction enzyme sites, and a potential for high copy number. Suitable expression vectors will be familiar to persons skilled in the art, illustrative examples of which include cloning vectors, modified cloning vectors, plasmids and viruses. Expression vectors that are capable of providing suitable levels of polypeptide expression in different hosts are also well known in the art. The choice of the vector will typically depend on the compatibility of the vector with the host cell into which the vector is to be introduced.

The present disclosure also extends to a host cell comprising the nucleic acid sequence described herein. The host cell may be transformed, transfected or transduced in a transient or stable manner. The nucleic acid, expression cassette or vector is introduced into a host cell so that the nucleic acid, cassette or vector is maintained as a chromosomal integrant or as a self-replicating extra-chromosomal vector. The term “host cell” encompasses any progeny of a parent host cell that is not identical to the parent host cell due to mutations that occur during replication. The host cell may be any cell useful in the production of a variant of the present invention, e.g., a prokaryote or a eukaryote. The prokaryotic host cell may be any Gram-positive or Gram-negative bacterium. The host cell may also be a eukaryotic cell, such as a yeast, fungal, mammalian, insect or plant cell. In a particular embodiment, the host cell is selected from the group of Escherichia coli, Pseudomonas, Bacillus, Streptomyces, Trichoderma, Aspergillus, Saccharomyces, Pichia, Thermus or Yarrowia.

The nucleic acid, expression cassette or expression vector according to the invention may be introduced into the host cell by any suitable method known to persons skilled in the art, illustrative examples of which include electroporation, conjugation, transduction, competent cell transformation, protoplast transformation, protoplast fusion, biolistic “gene gun” transformation, PEG-mediated transformation, lipid-assisted transformation or transfection, chemically mediated transfection, lithium acetate-mediated transformation and liposome-mediated transformation.

In an embodiment, the host cell is a genetically modified host cell or microorganism. In this context, a host cell or microorganism may be genetically modified to enhance the expression of the polypeptide in which it is expressed and/or PETase activity of the host cell. For example, the polypeptide described herein may be used to complement a wild type strain of a fungus or bacteria known to be capable of PETase activity, in order to improve and/or increase the PETase activity of that strain.

The present disclosure also extends to a method of producing a polypeptide having esterase activity, the method comprising:
(a) providing the polynucleotide, as described herein;
(b) expressing the polynucleotide in a host cell culture, thereby producing the polypeptide; and
(c) collecting the polypeptide produced in (b) from the host cell culture.

The present invention disclosure also extends to in vitro methods of producing the polypeptide described herein, the method comprising (a) contacting a nucleic acid, cassette or vector of the invention with an in vitro expression system; and (b) recovering the polypeptide produced. In vitro expression systems are well-known by the person skilled in the art and are commercially available.

Suitable host cells will be familiar to persons skilled in the art, illustrative examples of which include a recombinant Bacillus, recombinant E. coli, recombinant Pseudomonas, recombinant Aspergillus, recombinant Trichoderma, recombinant Streptomyces, recombinant Saccharomyces, recombinant Pichia, recombinant Thermus or recombinant Yarrowia. In an embodiment, the host cell is an E. coli.

The host cells may be cultivated in a nutrient medium suitable for production of polypeptides, using methods that will be known to persons skilled in the art. Suitable examples include cultivating the host cells by shake flask cultivation, or small-scale or large-scale fermentation (including continuous, batch, fed-batch, or solid state fermentations) in laboratory or industrial fermentors performed in a suitable medium and under conditions allowing the enzyme to be expressed and/or isolated. The cultivation will typically take place in a suitable nutrient medium, from commercial suppliers or prepared according to published compositions (e.g., in catalogues of the American Type Culture Collection) or any other culture medium suitable for cell growth.

Where the polypeptide is secreted into the nutrient medium, the polypeptide can be recovered directly from the culture supernatant. Conversely, the polypeptide can be recovered from cell lysates or after permeabilisation of the host cell membrane. The polypeptide may be recovered using any suitable method known to persons skilled in the art, illustrative examples of which include collection, centrifugation, filtration, extraction, spray-drying, evaporation, or precipitation. Optionally, the polypeptide may be partially or totally purified by a variety of procedures known in the art including, but not limited to, thermal shock, chromatography (e.g., ion exchange, affinity, hydrophobic, chromatofocusing, and size exclusion), electrophoretic procedures (e.g., preparative isoelectric focusing), differential solubility (e.g., ammonium sulfate precipitation), SDS-PAGE, or extraction to obtain substantially pure polypeptides.

The polypeptide may be used, in purified form, either alone or in combination with additional enzymes (e.g., PETases or MHETases or cutinases having PETase activity), to catalyze enzymatic reactions involved in the degradation and/or recycling of a polyester containing material, such as plastic products containing polyester. The polypeptides described herein may be in soluble form, or on solid phase. In particular, they may be bound to cell membranes or lipid vesicles, or to synthetic supports such as glass, plastic, polymers, filter, membranes, e.g., in the form of beads, columns, plates and the like.

The present disclosure also extends to compositions comprising the polypeptide, the nucleic acid or the host cell described herein.

The composition may be liquid or dry, for instance in the form of a powder. In some embodiments, the composition is a lyophilisate. For instance, the composition may comprise the polypeptide, nucleic acid and/or host cells and optionally excipients and/or reagents etc. Suitable excipients may include buffers commonly used in biochemistry, agents for adjusting pH, preservatives such as sodium benzoate, sodium sorbate or sodium ascorbate, conservatives, protective or stabilizing agents such as starch, dextrin, arabic gum, salts, sugars e.g., sorbitol, trehalose or lactose, glycerol, polyethyleneglycol, polyethene glycol, polypropylene glycol, propylene glycol, divalent ions such as calcium, sequestering agent such as EDTA, reducing agents (e.g., beta-mercaptoethanol, dithiothreitol, ascorbic acid, tris(2-carboxyethyl)phosphine), amino acids, a carrier such as a solvent or an aqueous solution, and the like.

In an embodiment, the composition comprises the polypeptide described herein (the polypeptide may be present in the composition in an isolated or at least partially purified form). In an embodiment, the composition comprises the polypeptide described herein in an amount of from about 0.1% to about 99.9%, preferably from about 0.1% to about 50%, preferably from about 0.1% to about 30%, preferably from about 0.1% to about 5% by weight of the total weight of the composition. In a preferred embodiment, the composition comprises the polypeptide described herein in an amount of from about 0.1 to about 5% by weight of the total weight of the composition. In another embodiment, the composition comprises the polypeptide described herein in an amount of from about 0.1 to about 0.2% by weight of the total weight of the composition. The amount of polypeptide in the composition may suitably adapted by persons skilled in the art, depending e.g., on the nature and/or amount of the polyester containing material to be degraded (hydrolysed) and/or the presence or absence of any additional enzymes/polypeptides in the composition.

The compositions described herein may further comprise additional polypeptide(s) exhibiting enzymatic activity, not limited to PETase, esterase, MHETase, or cutinase with promiscuous PETase activities.

In an embodiment, the polypeptide described herein is solubilized in an aqueous medium together with one or more excipients, such as excipients that may suitably stabilize or protect the polypeptide from degradation. For example, the polypeptides described herein may be solubilized in water and then admixed with excipients such as glycerol, sorbitol, dextrin, starch, glycol such as propanediol, salt, etc. The resulting admixture may then be dried so as to obtain a powder. Methods for drying such mixture are well known to the one skilled in the art and include, without limitation, lyophilisation, freeze-drying, spray-drying, supercritical drying, down-draught evaporation, thin-layer evaporation, centrifugal evaporation, conveyer drying, fluidized bed drying, drum drying or any combination thereof.

In an embodiment, the composition comprises at least one host cell expressing the polypeptide described herein, or an extract thereof. By “extract of a cell” is meant any fraction obtained from a cell, such as cell supernatant, cell debris, cell walls, DNA extract, enzymes or enzyme preparation or any preparation derived from cells by chemical, physical and/or enzymatic treatment, which is essentially free of living cells. Preferred extracts are enzymatically-active extracts. The composition may comprise one or several host cells or extract thereof containing the polypeptide described herein, and optionally one or several additional cells.

As noted elsewhere herein, the present inventors have surprisingly found that the polypeptides described herein (the engineered polypeptide and its variants) have greater esterase activity, that is the hydrolysis of mono-, di- and poly-terephthalic acid esters when compared to the extant PETases and cutinases with PETase activity. Particularly, the polypeptides described herein have improved hydrolase activity against mono- and di-terephthalic acid esters. Thus, disclosed herein is a method of hydrolysing mono-, di-, and poly-terephthalic acid esters, the method comprising exposing the terephthalic acid esters to the polypeptide, the composition or the host cell described herein, under conditions sufficient to convert the terephthalic acid esters to the terephthalic acid and ethylene glycol. The present disclosure also extends to a method of degrading a plastic product comprising a polyester, the method comprising exposing the plastic product to the polypeptide, the composition or the host cell described herein.

The present disclosure extends to the use of the polypeptide, the composition or the host cell described herein for degrading a polyester in aerobic or anaerobic conditions and/or recycling polyester containing material, as plastic products made of or containing polyesters and/or producing biodegradable plastic products containing polyester. Such methods and uses are particularly useful for degrading a plastic product comprising PET.

Advantageously, the polyester(s) of the polyester containing material is (are) depolymerized up to monomers and/or oligomers. In an embodiment, at least one polyester is degraded to yield re-polymerizable monomers and/or oligomers, which are advantageously retrieved or recovered for further use.

As noted elsewhere herein, the plastic product may comprise at least one polyester selected from the group consisting of polylactic acid (PLA), polytrimethylene terephthalate (PTT), polybutylene terephthalate (PBT), polyethylene isosorbide terephthalate (PEIT), polyethylene terephthalate (PET), polyhydroxyalkanoate (PHA), polybutylene succinate (PBS), polybutylene succinate adipate (PBSA), polybutylene adipate terephthalate (PBAT), polyethylene furanoate (PEF), polycaprolactone (PCL), poly(ethylene adipate) (PEA) and combinations of any of the foregoing.

The time required for degrading a polyester containing material may vary depending on the polyester containing material itself (i.e., nature and origin of the plastic product, its composition, shape etc.), the type and amount of polypeptide used, as well as various process parameters (i.e., temperature, pH, additional agents, etc.). One skilled in the art may easily adapt the process parameters to the polyester containing material.

Advantageously, the degrading process is implemented at a temperature from about 10° C. to about 80° C., preferably from about 20° C. to about 80° C., preferably from about 30° C. to about 80° C., preferably from about 40° C. to about 80° C., preferably from about 50° C. to about 80° C., even preferably from about 60° C. to about 80° C., even more preferably at about 60° C. to about 70° C., even more preferably at about 60° C. As the skilled person will appreciate, the temperature is typically maintained at an activating temperature, which corresponds to the temperature at which the polypeptide is active and/or the recombinant microorganism does synthesize, produce or release the polypeptide described herein. In an embodiment, the temperature is maintained below the glass transition temperature (Tg) of the polyester in the polyester containing material. In an embodiment, the degrading process or method is implemented at a temperature from about 10° C. to about 80° C., preferably from about 20° C. to about 80° C., preferably from about 30° C. to about 80° C., preferably from about 40° C. to about 80° C., preferably from about 50° C. to about 80° C., even preferably from about 60° C. to about 80° C., even more preferably at about 60° C. to about 70° C., even more preferably at about 60° C. The process or method may suitably be implemented in a continuous way, at a temperature at which the polypeptide can be used several times and/or recycled.

Advantageously, the degrading process or method is implemented at a pH in a range from about 5 to about 11, preferably in a range from about 6 to about 10, preferably at a pH from about 6.5 to about 9, preferably in a range from about 7 to about 9, preferably in a range from about 7 to about 8, preferably at a pH from about 9.5 to about 11.

In an embodiment, the polyester containing material may be pre-treated prior to be contacted with the polypeptide in order to physically change its structure, so as to increase the surface of contact between the polyester and the enzyme.

Monomers resulting from the depolymerization or degradation process or method may be suitably recovered, sequentially or continuously. A single type of monomers or several different types of monomers may be recovered, depending on the starting polyester containing material.

The recovered monomers may be further purified, using any suitable purifying method and conditioned in a repolymerizable form. Illustrative examples of suitable purifying methods include stripping process, separation by aqueous solution, steam selective condensation, filtration and concentration of the medium after the bioprocess, separation, distillation, vacuum evaporation, extraction, electrodialysis, adsorption, ion exchange, precipitation, crystallization, concentration and acid addition dehydration and precipitation, nanofiltration, acid catalyst treatment, semi continuous mode distillation or continuous mode distillation, solvent extraction, evaporative concentration, evaporative crystallization, liquid/liquid extraction, hydrogenation, azeotropic distillation process, adsorption, column chromatography, simple vacuum distillation and microfiltration, combined or not.

The repolymerizable monomers may be used to synthesize new polyesters. Advantageously, polyesters of same nature are repolymerized. However, it is possible to mix the recovered monomers with other monomers, for example, in order to synthesize new copolymers. Alternatively, the recovered monomers may be used as chemical intermediates in order to produce new chemical compounds of interest.

The present disclosure also extends to a composition comprising a plastic compound and the polypeptide, and/or host cell expressing said polypeptide or an extract thereof containing said polypeptide.

The present disclosure also extends to a masterbatch composition comprising the polypeptide, composition and/or host cell expressing said polypeptide or an extract thereof containing said polypeptide.

Advantageously, such plastic compound or masterbatch composition described herein can be used for the production of a polyester containing material.

In an embodiment, the resulting plastic compound or masterbatch composition is a biodegradable plastic compound or masterbatch composition complying with at least one of the relevant standards and/or labels known by the person skilled in the art, such as standard EN 13432, standard ASTM D6400, OK Biodegradation Soil (Label Vincotte), OK Biodegradation Water (Label Vincotte), OK Compost (Label Vincotte), OK Home Compost (Label Vincotte).

Advantageously, the degrading process of the polyester containing material (i.e., plastic compound or masterbatch composition or plastic product) is implemented at a temperature from about 10° C. to about 80° C., preferably from about 20° C. to about 80° C., preferably from about 30° C. to about 80° C., preferably from about 40° C. to about 80° C., preferably from about 50° C. to about 80° C., even preferably from about 60° C. to about 80° C., even more preferably from about 60° C. to about 70° C., even more preferably at about 60° C. +/−5° C.

Alternatively, the degrading process of the polyester containing material (i.e., plastic compound, masterbatch composition or plastic product) is implemented at a temperature from about 50° C. to about 70° C., more preferably at 60° C., +/−5° C.

The engineered polypeptides having esterase activity disclosed herein are suitable for a range of application, including industrial applications, illustrative examples of which include as additives in detergents, feed compositions (including for animal feed), textiles production, electronics and biomedical applications. For example, the engineered polypeptide having esterase activity disclosed herein can be employed in textile production, where it can be used as an exoesterase to suitably modify the properties of textile fibres.

The invention will now be described with reference to the following Examples which illustrate some preferred aspects of the present invention. However, it is to be understood that the particularity of the following description of the invention is not to supersede the generality of the preceding description of the invention.

### EXAMPLES

### Example 1: Ancestral Sequence Reconstruction

All amino acid sequences belonging to the protein families PF12695, PF01738 and PF12740 were retrieved from pfam. Redundancy was removed to 100% sequence identity using CD-HIT (Fu et al. 2012) and an all-vs-all pBLAST was performed. The resulting table was visualised as a sequence similarity network in cytoscape (Shannon et al. 2003) and edges connecting sequences with less than 40% sequence identity were deleted before the network was visualised as a prefuse-force directed network graph. All sequences belonging to the sequence cluster that included LCC, TfCut2 and IsPETase were retrieved from uniprot and redundancy was removed to 95% sequence identity using CD-HIT. Signal peptides for Gram-positive and negative bacteria were identified with SignalP5.0 (Almagro Armenteros et al. 2019) and manually removed. A multiple sequence alignment of this dataset was produced using the GINSI protocol in MAFFT (Katoh and Standley 2013), which was manually edited according to solved crystal structures. The final alignment had 397 aligned sequences.

100 independent replicates of maximum likelihood (ML) tree search and phylogenetic reconstruction were performed in IQ-TREE (Minh et al. 2020) using default tree search parameters. The sequence evolution model of best-fit (WAG+F+R8) (Whelan and Goldman 2001) was identified by Akaike and Bayesian information criteria in ModelFinder (Kalyaanamoorthy et al. 2017). Branch supports for each inference were calculated as alternate likelihood ratio test statistics (Anisimova and Gascuel 2006) and ultrafast bootstrap approximation (ufboot) (Hoang et al. 2018) to 1000 replicates. Because the approximately unbiased (AU) (Shimodaira 2002) test conducted to 10000 replicates failed to reject any single topology as statistically less likely than any other, 20 converged topologies that represented most of the topological diversity within the full dataset were sampled for ASR. Ancestral sequences were reconstructed over the 20 topologies by ML in CodeML from the PAML (Yang 2007) suite using the sequence evolution model WAG+G4. 18 conserved insertions that had been identified in the extant sequences were treated as discrete binomial traits (1 being the insertion is present, 0 being the insertion is absent) and were reconstructed by ML in the R package Ape (Paradis, Claude, and Strimmer 2004) using a binary Jukes-Cantor-like equal rates model. 48 reconstructed ancestral sequences were sampled from common nodes shared between LCC and TfCut2 over the 20 topologies that had a minimum ufboot support of 0.9 and a minimum mean posterior probability of 0.8.

Alternate reconstructions of two ancestral sequences (AncG4 and AncD3) (see Table 1) that exhibited PET hydrolytic activity in preliminary testing were additionally tested to account for reconstruction uncertainty. Residues that were reconstructed with an alternate amino acid that had a posterior probability greater than or equal to 0.2 were considered ambiguous. All 107 ambiguous single mutants (mutation to the less likely yet still statistically possible amino acid at each ambiguous site) of AncD3 and AncG4 were synthesized and experimentally characterised.

### Example 2: Protein Expression and Purification

Plasmids were transformed by heat shock into chemically competent E. Cloni® cells (Lucigen) and plated onto Lysogeny broth (LB) agar supplemented with 100 μg/L kanamycin and incubated at 37° C. overnight. A single colony was used to inoculate 1.5 mL autoinduction media supplemented with 100 μg/mL kanamycin in a 2.2 mL 96-well deep well block and grown at 1050 rpm at 37° C. for 5 hours, followed by room temperature (RT; 25° C.) for 16 hours.

Cells were harvested by centrifugation at 2000×g for 15 minutes at RT and resuspended in Lysis Buffer (1× BugBuster® Protein Extraction Reagent (Merck-Millipore), 20 mM Tris, 300 mM NaCl, 1 U/ml Turbonuclease (Sigma) pH 8). The cell suspension was left to incubate at RT for 20 minutes with gentle shaking. The lysate was separated from the insoluble cell debris by centrifugation at 2250×g for 1 hour at RT and successful expression of the enzyme was confirmed.

The clarified lysate was then diluted with 100 μl of Equilibration Buffer (20 mM Tris, 300 mM NaCl pH 8) and purified by nickel-charged IMAC using a 96-well HisPur™ Ni-NTA Spin Plate (ThermoFisher Scientific) equilibrated in Equilibration Buffer, washing the sample three times with 250 μl of Wash Buffer (20 mM Tris, 300 mM NaCl, 10 mM imidazole pH 8) and eluting with 250 μl of Elution Buffer (20 mM Tris, 300 mM NaCl, 150 mM imidazole pH 8). All centrifugation steps following addition of Wash or Elution Buffer were at 1000×g for 1 minute at RT. The eluate was stored at 4° C.

### Example 3: Large Scale Protein Expression and Purification

Plasmids were transformed by electroporation into electrocompetent E. Cloni® cells (Lucigen) and plated onto Lysogeny broth (LB) agar supplemented with 100 μg/L kanamycin and incubated at 37° C. overnight. A single colony was used to inoculate 10 mL of LB supplemented with 100 μg/mL kanamycin and grown at 37° C. overnight at 180 rpm. The 10 mL culture was used to inoculate 1 L of autoinduction media supplemented with 100 μg/mL kanamycin and grown at RT for 23 hours.

Cells were harvested by centrifugation at 5000×g for 15 minutes at 4° C. and the cell pellet was stored at −20° C. until purification. To purify, the cell pellet was resuspended in Buffer A (50 mM Tris, 25 mM imidazole pH 8) and 1 U/ml Turbonuclease (Sigma). The cell suspension was lysed by two rounds of sonication at 50% power and pulse time of 5 minutes. Soluble cell lysate was separated from the insoluble cell debris by centrifugation at 13,000 rpm for 60 minutes at 4° C. The lysate was filtered using a 0.45 μm pore size filter and then purified by nickel-charged IMAC using a 5 mL HisTrap HP (GE Healthcare Life Sciences) equilibrated in Buffer A and eluted with Buffer B (50 mM Tris, 500 mM imidazole pH 8). The eluate was collected and filtered using a 0.2 μm pore size filter and further purified using a HiLoad 26/600 Superdex 200 (GE Healthcare Life Sciences) equilibrated in SEC Buffer (20 mM HEPES, 150 mM NaCl pH 7.5). The recombinant enzymes were confirmed to be suitably expressed.

### Example 4: UV Absorbance Assay to Measure Activity and Kinetics of Candidate Proteins

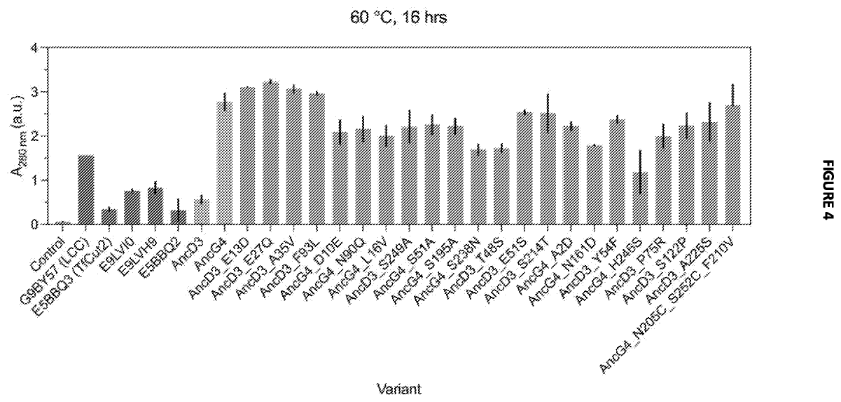

For purified protein from 96-well expression and purification, 15 μl of the eluate from the 96-well Ni-NTA purification and 285 μl of Reaction Buffer (50 mM Bicine pH 9) was added to a clear 96-well plate. For purified protein from large-scale expression and purification, 300 μl of 100 nM enzyme in Reaction Buffer was added to a clear 96-well plate. A single disk of amorphous PET (Goodfellow ES301445) with 4 mm diameter and 0.25 mm thickness was added to each well. The plate was incubated at 60° C. for 16 hours. Following incubation, 100 μl of the reaction solution was transferred to a clear UV-transparent 96-well plate and the absorbance was measured between 240 to 300 nm in 1 nm steps using the Epoch Microplate Spectrophotometer (BioTek). For comparison of activity of all variants, the absorbance at 270 nm was used (see FIG. 4).

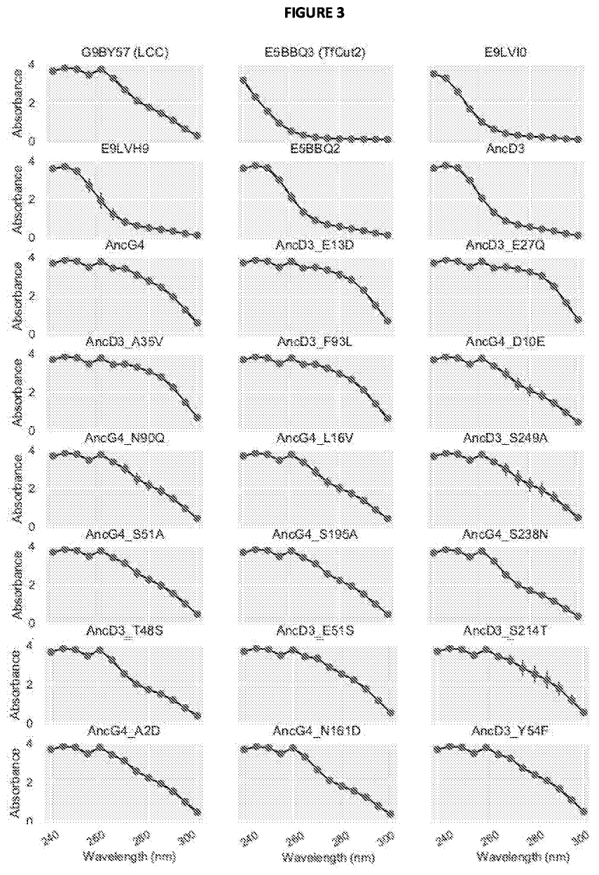

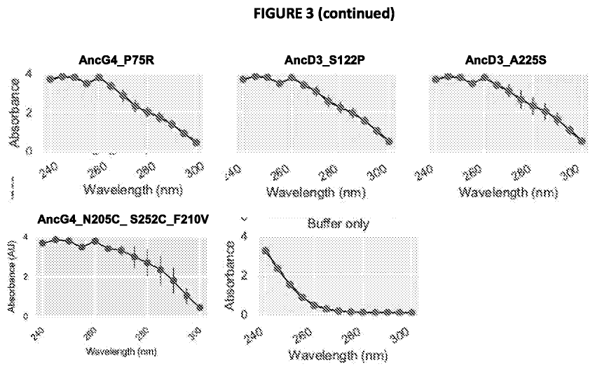

A 150 μl reaction containing 100 nM enzyme, 50 mM Bicine pH 9 and 1 disk of amorphous PET (Goodfellow ES301445) with 4 mm diameter and 0.25 mm thickness was prepared. The reaction was incubated at 50, 60, 70, or 80° C. for 4 hours. Following incubation, 100 μl of the reaction solution was transferred to a clear UV-transparent 96-well plate and the absorbance was measured between 240 to 300 nm in 1 nm steps using the Epoch Microplate Spectrophotometer (BioTek) (see FIG. 3). For comparison of activity of all variants, the absorbance at 270 nm was used. Selected variants and their amino acid sequences are shown in Table 1.

### Example 5: Differential Scanning Fluorimetry and Thermal Stability

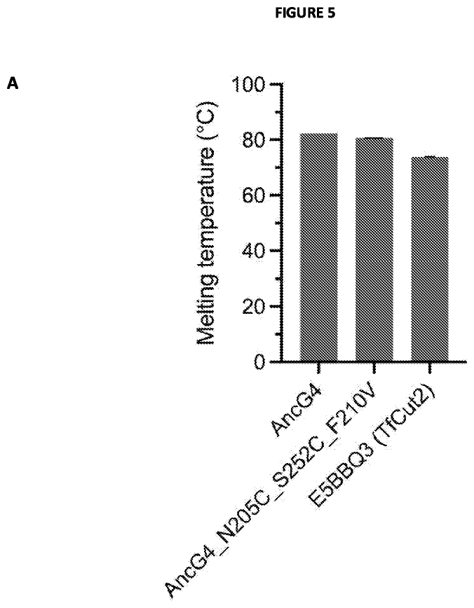

A 20 μl reaction containing 1× Protein Thermal Shift™ Dye (ThermoFisher Scientific), lx Protein Thermal Shift™ Buffer (ThermoFisher Scientific) and 10 μM of purified enzyme was prepared and transferred to a MicroAmp™ EnduraPlate™ Optical 96-Well Clear Reaction plate (ThermoFisher Scientific). The QuantStudio 3 Real-Time PCR System (ThermoFisher Scientific) was used to measure fluorescence as the sample was heated from 25 to 90° C. at a rate of 0.05° C./second. The data were analysed using the Protein Thermal Shift™ Software and a Boltzmann curve was fitted to the data to determine the Tm. As shown in FIG. 5, the melting temperature (Tm) of AncG4 is about 82.4° C., and AncG4_N205C_S252C_F210V is about 80.6° C., when compared to extant cutinase E5BBQ3 (TfCut2) (73.9° C.), as determined by differential scanning fluorimetry (DSF).

### REFERENCES

Almagro Armenteros, Jose Juan, Konstantinos D. Tsirigos, Casper Kaae Sønderby, Thomas Nordahl Petersen, Ole Winther, Søren Brunak, Gunnar von Heijne, and Henrik Nielsen. 2019. “SignalP 5.0 Improves Signal Peptide Predictions Using Deep Neural Networks.” Nature Biotechnology 37 (4): 420-23.
Anisimova, M., and O. Gascuel. 2006. “Approximate Likelihood-Ratio Test for Branches: A Fast, Accurate, and Powerful Alternative.” Systematic Biology 55 (4). https://doi.org/10.1080/10635150600755453.
Austin, Harry P., Mark D. Allen, Bryon S. Donohoe, Nicholas A. Rorrer, Fiona L. Kearns, Rodrigo L. Silveira, Benjamin C. Pollard, et al. 2018. “Characterization and Engineering of a Plastic-Degrading Aromatic Polyesterase.” Proceedings of the National Academy of Sciences of the United States of America 115 (19): E4350-57.
Castro-Ochoa, D., C. Peña-Montes, A. González-Canto, A. Alva-Gasca, R. Esquivel-Bautista, A. Navarro-Ocaña, and A. Farrés. 2012. “ANCUT2, an Extracellular Cutinase from Aspergillus Nidulans Induced by Olive Oil.” Applied Biochemistry and Biotechnology 166 (5). https://doi.org/10.1007/s12010-011-9513-7.
“Chapter 4 Cutinases: Properties and Industrial Applications.” 2009. In Advances in Applied Microbiology, 66:77-95. Academic Press.
Cui, Yinglu, Yanchun Chen, Xinyue Liu, Saijun Dong, Yu'e Tian, Yuxin Qiao, Ruchira Mitra, et al. 2021. “Computational Redesign of a PETase for Plastic Biodegradation under Ambient Condition by the GRAPE Strategy.” ACS Catalysis. https://doi.org/10.1021/acscatal.0c05126.
“Cutinases from Thermophilic Bacteria (actinomycetes): From Identification to Functional and Structural Characterization.” 2021. In Methods in Enzymology, 648:159-85. Academic Press.
Ettinger, William F., Sushil K. Thukral, and Pappachan E. Kolattukudy. 1987. “Structure of Cutinase Gene, cDNA, and the Derived Amino Acid Sequence from Phytopathogenic Fungi.” Biochemistry. https://doi.org/10.1021/bi00398a052.
Fu, Limin, Beifang Niu, Zhengwei Zhu, Sitao Wu, and Weizhong Li. 2012. “CD-HIT: Accelerated for Clustering the next-Generation Sequencing Data.” Bioinformatics 28 (23): 3150-52.
Furukawa, Makoto, Norifumi Kawakami, Atsushi Tomizawa, and Kenji Miyamoto. 2019. “Efficient Degradation of Poly(ethylene Terephthalate) with Thermobifida Fusca Cutinase Exhibiting Improved Catalytic Activity Generated Using Mutagenesis and Additive-Based Approaches.” Scientific Reports 9 (1): 16038.
Furukawa, Ryutaro, Wakako Toma, Koji Yamazaki, and Satoshi Akanuma. 2020. “Ancestral Sequence Reconstruction Produces Thermally Stable Enzymes with Mesophilic Enzyme-like Catalytic Properties.” Scientific Reports 10 (1): 15493.
Gemeren, I. A. van, A. Beijersbergen, C. A. M. J. J. van den Hondel, and C. T. Verrips. 1998. “Expression and Secretion of Defined Cutinase Variants by Aspergillus Awamori.” Applied and Environmental Microbiology 64 (8): 2794.
Gumulya, Yosephin, Jong-Min Baek, Shun-Jie Wun, Raine E. S. Thomson, Kurt L. Harris, Dominic J. B. Hunter, James B. Y. Behrendorff, et al. 2018. “Engineering Highly Functional Thermostable Proteins Using Ancestral Sequence Reconstruction.” Nature Catalysis. https://doi.org/10.1038/s41929-018-0159-5.
Hoang, Diep Thi, Olga Chernomor, Arndt von Haeseler, Bui Quang Minh, and Le Sy Vinh. 2018. “UFBoot2: Improving the Ultrafast Bootstrap Approximation.” Molecular Biology and Evolution 35 (2): 518-22.
Kalyaanamoorthy, Subha, Bui Quang Minh, Thomas K. F. Wong, Arndt von Haeseler, and Lars S. Jermiin. 2017. “ModelFinder: Fast Model Selection for Accurate Phylogenetic Estimates.” Nature Methods 14 (6): 587-89.
Katoh, Kazutaka, and Daron M. Standley. 2013. “MAFFT Multiple Sequence Alignment Software Version 7: Improvements in Performance and Usability.” Molecular Biology and Evolution 30 (4): 772-80.
Kleeberg, I., K. Welzel, J. Vandenheuvel, R-J Müller, and W-D Deckwer. 2005. “Characterization of a New Extracellular Hydrolase from Thermobifida Fusca Degrading Aliphatic-Aromatic Copolyesters.” Biomacromolecules 6 (1): 262-70.
Martinez, Chrislaine, Pieter De Geus, Marc Lauwereys, Gaston Matthyssens, and Christian Cambillau. 1992. “Fusarium Solani Cutinase Is a Lipolytic Enzyme with a Catalytic Serine Accessible to Solvent.” Nature 356 (6370): 615-18.
Ma, Yuan, Mingdong Yao, Bingzhi Li, Mingzhu Ding, Bo He, Si Chen, Xiao Zhou, and Yingjin Yuan. 2018. “Enhanced Poly(ethylene Terephthalate) Hydrolase Activity by Protein Engineering.” Engineering. https://doi.org/10.1016/j.eng.2018.09.007.
Minh, Bui Quang, Heiko A. Schmidt, Olga Chernomor, Dominik Schrempf, Michael D. Woodhams, Arndt von Haeseler, and Robert Lanfear. 2020. “Corrigendum to: IQ-TREE 2: New Models and Efficient Methods for Phylogenetic Inference in the Genomic Era.” Molecular Biology and Evolution 37 (8): 2461.
Nyyssölä, Antti. 2015. “Which Properties of Cutinases Are Important for Applications?” Applied Microbiology and Biotechnology 99 (12): 4931-42.
Paradis, E., J. Claude, and K. Strimmer. 2004. “APE: Analyses of Phylogenetics and Evolution in R Language.” Bioinformatics. https://doi.org/10.1093/bioinformatics/btg412.
Perez-Garcia, Pablo, Stefanie Kobus, Christoph G. W. Gertzen, Astrid Hoeppner, Nicholas Holzscheck, Christoph Heinrich Strunk, Harald Huber, et al. 2021. “A Promiscuous Ancestral Enzyme's Structure Unveils Protein Variable Regions of the Highly Diverse Metallo-β-Lactamase Family.” Communications Biology 4 (1): 132.
Ribitsch, Doris, Enrique Herrero Acero, Katrin Greimel, Inge Eiteljoerg, Eva Trotscha, Giuliano Freddi, Helmut Schwab, and Georg M. Guebitz. 2012. “Characterization of a New Cutinase from Thermobifida Albafor PET-Surface Hydrolysis.” Biocatalysis and Biotransformation. https://doi.org/10.3109/10242422.2012.644435.
Ribitsch, Doris, Antonio Orcal Yebra, Sabine Zitzenbacher, Jing Wu, Susanne Nowitsch, Georg Steinkellner, Katrin Greimel, et al. 2013. “Fusion of Binding Domains to Thermobifida cellulosilytica Cutinase to Tune Sorption Characteristics and Enhancing PET Hydrolysis.” Biomacromolecules 14 (6): 1769-76.
Roth, Christian, Ren Wei, Thorsten Oeser, Johannes Then, Christina Füllner, Wolfgang Zimmermann, and Norbert Striiter. 2014. “Structural and Functional Studies on a Thermostable Polyethylene Terephthalate Degrading Hydrolase from Thermobifida fusca.” Applied Microbiology and Biotechnology 98 (18): 7815-23.
Shannon, Paul, Andrew Markiel, Owen Ozier, Nitin S. Baliga, Jonathan T. Wang, Daniel Ramage, Nada Amin, Benno Schwikowski, and Trey Ideker. 2003. “Cytoscape: A Software Environment for Integrated Models of Biomolecular Interaction Networks.” Genome Research 13 (11): 2498-2504.
Shi, Lixia, Haifeng Liu, Songfeng Gao, Yunxuan Weng, and Leilei Zhu. 2021. “Enhanced Extracellular Production of IsPETase in Escherichia coli via Engineering of the pelB Signal Peptide.” Journal of Agricultural and Food Chemistry. https://doi.org/10.1021/acs.jafc.007469.
Shimodaira, Hidetoshi. 2002. “An Approximately Unbiased Test of Phylogenetic Tree Selection.” Systematic Biology 51 (3): 492-508.
Shirke, Abhijit N., Christine White, Jacob A. Englaender, Allison Zwarycz, Glenn L. Butterfoss, Robert J. Linhardt, and Richard A. Gross. 2018. “Stabilizing Leaf and Branch Compost Cutinase (LCC) with Glycosylation: Mechanism and Effect on PET Hydrolysis.” Biochemistry. https://doi.org/10.1021/acs.biochem.7b01189.
Silva, Carla, Shi Da, Nádia Silva, Teresa Matamá, Rita Araújo, Madalena Martins, Sheng Chen, et al. 2011. “Engineered Thermobifida Fusca Cutinase with Increased Activity on Polyester Substrates.” Biotechnology Journal 6 (10): 1230-39.
Son, Hyeoncheol Francis, In Jin Cho, Seongjoon Joo, Hogyun Seo, Hye-Young Sagong, So Young Choi, Sang Yup Lee, and Kyung-Jin Kim. 2019. “Rational Protein Engineering of Thermo-Stable PETase from Ideonella Sakaiensis for Highly Efficient PET Degradation.” ACS Catalysis. https://doi.org/10.1021/acscatal.9b00568.
Son, Hyeoncheol Francis, Seongjoon Joo, Hogyun Seo, Hye-Young Sagong, Seul Hoo Lee, Hwaseok Hong, and Kyung-Jin Kim. 2020. “Structural Bioinformatics-Based Protein Engineering of Thermo-Stable PETase from Ideonella Sakaiensis.” Enzyme and Microbial Technology 141 (November): 109656.
Spence, Matthew A., Joe A. Kaczmarski, Jake W. Saunders, and Colin J. Jackson. 2021. “Ancestral Sequence Reconstruction for Protein Engineers.” Current Opinion in Structural Biology 69 (May): 131-41.
Sulaiman, Sintawee, Saya Yamato, Eiko Kanaya, Joong-Jae Kim, Yuichi Koga, Kazufumi Takano, and Shigenori Kanaya. 2012. “Isolation of a Novel Cutinase Homolog with Polyethylene Terephthalate-Degrading Activity from Leaf-Branch Compost by Using a Metagenomic Approach.” Applied and Environmental Microbiology 78 (5): 1556.
Sweigard, J. A., F. G. Chumley, and B. Valent. 1992. “Cloning and Analysis of CUT1, a Cutinase Gene from Magnaporthe grisea.” Molecular & General Genetics: MGG 232 (2): 174-82.
Then, Johannes, Ren Wei, Thorsten Oeser, André Gerdts, Juliane Schmidt, Markus Barth, and Wolfgang Zimmermann. 2016. “A Disulfide Bridge in the Calcium Binding Site of a Polyester Hydrolase Increases Its Thermal Stability and Activity against Polyethylene Terephthalate.” FEBS Open Bio 6 (5): 425-32.
Thomas, Adam, Rhys Cutlan, William Finnigan, Mark van der Giezen, and Nicholas Harmer. 2019. “Highly Thermostable Carboxylic Acid Reductases Generated by Ancestral Sequence Reconstruction.” Communications Biology 2 (November): 429.
Tournier, V., C. M. Topham, A. Gilles, B. David, C. Folgoas, E. Moya-Leclair, E. Kamionka, et al. 2020. “An Engineered PET Depolymerase to Break down and Recycle Plastic Bottles.” Nature 580 (7802): 216-19.
Wei, Ren, Thorsten Oeser, Juliane Schmidt, René Meier, Markus Barth, Johannes Then, and Wolfgang Zimmermann. 2016. “Engineered Bacterial Polyester Hydrolases Efficiently Degrade Polyethylene Terephthalate due to Relieved Product Inhibition.” Biotechnology and Bioengineering 113 (8): 1658-65.
Wheeler, Lucas C., and Michael J. Harms. 2021. “Were Ancestral Proteins Less Specific?” Molecular Biology and Evolution 38 (6): 2227-39.
Whelan, S., and N. Goldman. 2001. “A General Empirical Model of Protein Evolution Derived from Multiple Protein Families Using a Maximum-Likelihood Approach.” Molecular Biology and Evolution 18 (5): 691-99.
Wilding, Matthew, Thomas S. Peat, Subha Kalyaanamoorthy, Janet Newman, Colin Scott, and Lars S. Jermiin. 2017. “Reverse Engineering: Transaminase Biocatalyst Development Using Ancestral Sequence Reconstruction.” Green Chemistry. https://doi.org/10.1039/c7gc02343j.
Yang, Ziheng. 2007. “PAML 4: Phylogenetic Analysis by Maximum Likelihood.” Molecular Biology and Evolution 24 (8): 1586-91.
Yoshida, Shosuke, Kazumi Hiraga, Toshihiko Takehana, Ikuo Taniguchi, Hironao Yamaji, Yasuhito Maeda, Kiyotsuna Toyohara, Kenji Miyamoto, Yoshiharu Kimura, and Kohei Oda. 2016. “A Bacterium That Degrades and Assimilates Poly(ethylene Terephthalate).” Science 351 (6278): 1196-99.

TABLE 1
Amino acid sequences of polypeptides disclosed herein

| SEQ | | |
|---|---|---|
| ID | | |
| NO: | Name | Sequence |
| 1 | Ancestral | MYERGPDPTEASLEASSGPFSVSETSVSRLSASGFGGG |
| | Sequence | TIYYPTTTSSGTYGAVAISPGYTATQSSIAWLGPRLASH |
| | Reconstruction | GFVVITIDTNTTFDQPDSRARQLMAALNYLVNRSSVRS |
| | G4 | RIDSSRLAVMGHSMGGGGTLRAAEDNPSLKAAIPLTP |
| | | WHTNKNWSSVRVPTLIIGAENDTIAPVSSHAKPFYNSL |
| | | PSSTPKAYLELNGASHFAPNSSNTTIGKYSVSWLKRFV |
| | | DNDTRYSQFLCPAPHDDSAISEYRSTCPY |
| 2 | Ancestral | MYERGPDPTESSLEASSGPFSVSETSVSRLSASGFGGGT |
| | Sequence | IYYPTTTSEGTYGAVAISPGYTATQSSIAWLGPRLASH |
| | Reconstruction | GFVVITIDTNTTFDQPDSRARQLMAALNYLVNRSSVRS |
| | D3 | RIDSSRLAVMGHSMGGGGTLRAAEDNPSLKAAIPLTP |
| | | WHTNKNWSSVRVPTLIIGAENDTIAPVSSHAKPFYNSL |
| | | PSSTPKAYLELNGASHFAPNSSNTTIGKYSIAWLKRFV |
| | | DNDTRYSQFLCPAPHDDSAISEYRSTCPY |
| 3 | AncG4 | AANPYERGPDPTEASLEASSGPFSVSETSVSRLSASGFG |
| | | GGTIYYPTTTSSGTYGAVAISPGYTATQSSIAWLGPRL |
| | | ASHGFVVITIDTNTTFDQPDSRARQLMAALNYLVNRSS |
| | | VRSRIDSSRLAVMGHSMGGGGTLRAAEDNPSLKAAIP |
| | | LTPWHTNKNWSSVRVPTLIIGAENDTIAPVSSHAKPFY |
| | | NSLPSSTPKAYLELNGASHFAPNSSNTTIGKYSVSWLK |
| | | RFVDNDTRYSQFLCPAPHDDSAISEYRSTCPY |
| 4 | AncD3 | AANPYERGPDPTESSLEASSGPFSVSETSVSRLSASGFG |
| | | GGTIYYPTTTSEGTYGAVAISPGYTATQSSIAWLGPRL |
| | | ASHGFVVITIDTNTTFDQPDSRARQLMAALNYLVNRSS |
| | | VRSRIDSSRLAVMGHSMGGGGTLRAAEDNPSLKAAIP |
| | | LTPWHTNKNWSSVRVPTLIIGAENDTIAPVSSHAKPFY |
| | | NSLPSSTPKAYLELNGASHFAPNSSNTTIGKYSIAWLK |
| | | RFVDNDTRYSQFLCPAPHDDSAISEYRSTCPY |
| 5 | AncG4_A2D | ADNPYERGPDPTEASLEASSGPFSVSETSVSRLSASGFG |
| | | GGTIYYPTTTSSGTYGAVAISPGYTATQSSIAWLGPRL |
| | | ASHGFVVITIDTNTTFDQPDSRARQLMAALNYLVNRSS |
| | | VRSRIDSSRLAVMGHSMGGGGTLRAAEDNPSLKAAIP |
| | | LTPWHTNKNWSSVRVPTLIIGAENDTIAPVSSHAKPFY |
| | | NSLPSSTPKAYLELNGASHFAPNSSNTTIGKYSVSWLK |
| | | RFVDNDTRYSQFLCPAPHDDSAISEYRSTCPY |
| 6 | AncG4_D10E | AANPYERGPEPTEASLEASSGPFSVSETSVSRLSASGFG |
| | | GGTIYYPTTTSSGTYGAVAISPGYTATQSSIAWLGPRL |
| | | ASHGFVVITIDTNTTFDQPDSRARQLMAALNYLVNRSS |
| | | VRSRIDSSRLAVMGHSMGGGGTLRAAEDNPSLKAAIP |
| | | LTPWHTNKNWSSVRVPTLIIGAENDTIAPVSSHAKPFY |
| | | NSLPSSTPKAYLELNGASHFAPNSSNTTIGKYSVSWLK |
| | | RFVDNDTRYSQFLCPAPHDDSAISEYRSTCPY |
| 7 | AncG4_L16V | AANPYERGPDPTEASVEASSGPFSVSETSVSRLSASGFG |
| | | GGTIYYPTTTSSGTYGAVAISPGYTATQSSIAWLGPRL |
| | | ASHGFVVITIDTNTTFDQPDSRARQLMAALNYLVNRSS |
| | | VRSRIDSSRLAVMGHSMGGGGTLRAAEDNPSLKAAIP |
| | | LTPWHTNKNWSSVRVPTLIIGAENDTIAPVSSHAKPFY |
| | | NSLPSSTPKAYLELNGASHFAPNSSNTTIGKYSVSWLK |
| | | RFVDNDTRYSQFLCPAPHDDSAISEYRSTCPY |
| 8 | AncG4_S51A | AANPYERGPDPTEASLEASSGPFSVSETSVSRLSASGFG |
| | | GGTIYYPTTTSAGTYGAVAISPGYTATQSSIAWLGPRL |
| | | ASHGFVVITIDTNTTFDQPDSRARQLMAALNYLVNRSS |
| | | VRSRIDSSRLAVMGHSMGGGGTLRAAEDNPSLKAAIP |
| | | LTPWHTNKNWSSVRVPTLIIGAENDTIAPVSSHAKPFY |
| | | NSLPSSTPKAYLELNGASHFAPNSSNTTIGKYSVSWLK |
| | | RFVDNDTRYSQFLCPAPHDDSAISEYRSTCPY |
| 9 | AncG4_N90Q | AANPYERGPDPTEASLEASSGPFSVSETSVSRLSASGFG |
| | | GGTIYYPTTTSSGTYGAVAISPGYTATQSSIAWLGPRL |
| | | ASHGFVVITIDTQTTFDQPDSRARQLMAALNYLVNRSS |
| | | VRSRIDSSRLAVMGHSMGGGGTLRAAEDNPSLKAAIP |
| | | LTPWHTNKNWSSVRVPTLIIGAENDTIAPVSSHAKPFY |
| | | NSLPSSTPKAYLELNGASHFAPNSSNTTIGKYSVSWLK |
| | | RFVDNDTRYSQFLCPAPHDDSAISEYRSTCPY |
| 10 | AncG4_N161D | AANPYERGPDPTEASLEASSGPFSVSETSVSRLSASGFG |
| | | GGTIYYPTTTSSGTYGAVAISPGYTATQSSIAWLGPRL |
| | | ASHGFVVITIDTNTTFDQPDSRARQLMAALNYLVNRSS |
| | | VRSRIDSSRLAVMGHSMGGGGTLRAAEDNPSLKAAIP |
| | | LTPWHTNKDWSSVRVPTLIIGAENDTIAPVSSHAKPFY |
| | | NSLPSSTPKAYLELNGASHFAPNSSNTTIGKYSVSWLK |
| | | RFVDNDTRYSQFLCPAPHDDSAISEYRSTCPY |
| 11 | AncG4_S195A | AANPYERGPDPTEASLEASSGPFSVSETSVSRLSASGFG |
| | | GGTIYYPTTTSSGTYGAVAISPGYTATQSSIAWLGPRL |
| | | ASHGFVVITIDTNTTFDQPDSRARQLMAALNYLVNRSS |
| | | VRSRIDSSRLAVMGHSMGGGGTLRAAEDNPSLKAAIP |
| | | LTPWHTNKNWSSVRVPTLIIGAENDTIAPVSSHAKPFY |
| | | NSLPASTPKAYLELNGASHFAPNSSNTTIGKYSVSWLK |
| | | RFVDNDTRYSQFLCPAPHDDSAISEYRSTCPY |
| 12 | AncG4_S238N | AANPYERGPDPTEASLEASSGPFSVSETSVSRLSASGFG |
| | | GGTIYYPTTTSSGTYGAVAISPGYTATQSSIAWLGPRL |
| | | ASHGFVVITIDTNTTFDQPDSRARQLMAALNYLVNRSS |
| | | VRSRIDSSRLAVMGHSMGGGGTLRAAEDNPSLKAAIP |
| | | LTPWHTNKNWSSVRVPTLIIGAENDTIAPVSSHAKPFY |
| | | NSLPSSTPKAYLELNGASHFAPNSSNTTIGKYSVSWLK |
| | | RFVDNDTRYNQFLCPAPHDDSAISEYRSTCPY |
| 13 | AncD3_E13D | AANPYERGPDPTDSSLEASSGPFSVSETSVSRLSASGFG |
| | | GGTIYYPTTTSEGTYGAVAISPGYTATQSSIAWLGPRL |
| | | ASHGFVVITIDTNTTFDQPDSRARQLMAALNYLVNRSS |
| | | VRSRIDSSRLAVMGHSMGGGGTLRAAEDNPSLKAAIP |
| | | LTPWHTNKNWSSVRVPTLIIGAENDTIAPVSSHAKPFY |
| | | NSLPSSTPKAYLELNGASHFAPNSSNTTIGKYSIAWLK |
| | | RFVDNDTRYSQFLCPAPHDDSAISEYRSTCPY |
| 14 | AncD3_E27Q | AANPYERGPDPTESSLEASSGPFSVSQTSVSRLSASGFG |
| | | GGTIYYPTTTSEGTYGAVAISPGYTATQSSIAWLGPRL |
| | | ASHGFVVITIDTNTTFDQPDSRARQLMAALNYLVNRSS |
| | | VRSRIDSSRLAVMGHSMGGGGTLRAAEDNPSLKAAIP |
| | | LTPWHTNKNWSSVRVPTLIIGAENDTIAPVSSHAKPFY |
| | | NSLPSSTPKAYLELNGASHFAPNSSNTTIGKYSIAWLK |
| | | RFVDNDTRYSQFLCPAPHDDSAISEYRSTCPY |
| 15 | AncD3_A35V | AANPYERGPDPTESSLEASSGPFSVSETSVSRLSVSGFG |
| | | GGTIYYPTTTSEGTYGAVAISPGYTATQSSIAWLGPRL |
| | | ASHGFVVITIDTNTTFDQPDSRARQLMAALNYLVNRSS |
| | | VRSRIDSSRLAVMGHSMGGGGTLRAAEDNPSLKAAIP |
| | | LTPWHTNKNWSSVRVPTLIIGAENDTIAPVSSHAKPFY |
| | | NSLPSSTPKAYLELNGASHFAPNSSNTTIGKYSIAWLK |
| | | RFVDNDTRYSQFLCPAPHDDSAISEYRSTCPY |
| 16 | AncD3_T48S | AANPYERGPDPTESSLEASSGPFSVSETSVSRLSASGFG |
| | | GGTIYYPTSTSEGTYGAVAISPGYTATQSSIAWLGPRL |
| | | ASHGFVVITIDTNTTFDQPDSRARQLMAALNYLVNRSS |
| | | VRSRIDSSRLAVMGHSMGGGGTLRAAEDNPSLKAAIP |
| | | LTPWHTNKNWSSVRVPTLIIGAENDTIAPVSSHAKPFY |
| | | NSLPSSTPKAYLELNGASHFAPNSSNTTIGKYSIAWLK |
| | | RFVDNDTRYSQFLCPAPHDDSAISEYRSTCPY |
| 17 | AncD3_E51S | AANPYERGPDPTESSLEASSGPFSVSETSVSRLSASGFG |
| | | GGTIYYPTTTSSGTYGAVAISPGYTATQSSIAWLGPRL |
| | | ASHGFVVITIDTNTTFDQPDSRARQLMAALNYLVNRSS |
| | | VRSRIDSSRLAVMGHSMGGGGTLRAAEDNPSLKAAIP |
| | | LTPWHTNKNWSSVRVPTLIIGAENDTIAPVSSHAKPFY |
| | | NSLPSSTPKAYLELNGASHFAPNSSNTTIGKYSIAWLK |
| | | RFVDNDTRYSQFLCPAPHDDSAISEYRSTCPY |
| 18 | AncD3_Y54F | AANPYERGPDPTESSLEASSGPFSVSETSVSRLSASGFG |
| | | GGTIYYPTTTSEGTFGAVAISPGYTATQSSIAWLGPRLA |
| | | SHGFVVITIDTNTTFDQPDSRARQLMAALNYLVNRSSV |
| | | RSRIDSSRLAVMGHSMGGGGTLRAAEDNPSLKAAIPL |
| | | TPWHTNKNWSSVRVPTLIIGAENDTIAPVSSHAKPFYN |
| | | SLPSSTPKAYLELNGASHFAPNSSNTTIGKYSIAWLKRF |
| | | VDNDTRYSQFLCPAPHDDSAISEYRSTCPY |
| 19 | AncD3_P75R | AANPYERGPDPTESSLEASSGPFSVSETSVSRLSASGFG |
| | | GGTIYYPTTTSEGTYGAVAISPGYTATQSSIAWLGRRL |
| | | ASHGFVVITIDTNTTFDQPDSRARQLMAALNYLVNRSS |
| | | VRSRIDSSRLAVMGHSMGGGGTLRAAEDNPSLKAAIP |
| | | LTPWHTNKNWSSVRVPTLIIGAENDTIAPVSSHAKPFY |
| | | NSLPSSTPKAYLELNGASHFAPNSSNTTIGKYSIAWLK |
| | | RFVDNDTRYSQFLCPAPHDDSAISEYRSTCPY |
| 20 | AncD3_F93L | AANPYERGPDPTESSLEASSGPFSVSETSVSRLSASGFG |
| | | GGTIYYPTTTSEGTYGAVAISPGYTATQSSIAWLGPRL |
| | | ASHGFVVITIDTNTTLDQPDSRARQLMAALNYLVNRSS |
| | | VRSRIDSSRLAVMGHSMGGGGTLRAAEDNPSLKAAIP |
| | | LTPWHTNKNWSSVRVPTLIIGAENDTIAPVSSHAKPFY |
| | | NSLPSSTPKAYLELNGASHFAPNSSNTTIGKYSIAWLK |
| | | RFVDNDTRYSQFLCPAPHDDSAISEYRSTCPY |
| 21 | AncD3_N108D | AANPYERGPDPTESSLEASSGPFSVSETSVSRLSASGFG |
| | | GGTIYYPTTTSEGTYGAVAISPGYTATQSSIAWLGPRL |
| | | ASHGFVVITIDTNTTFDQPDSRARQLMAALDYLVNRSS |
| | | VRSRIDSSRLAVMGHSMGGGGTLRAAEDNPSLKAAIP |
| | | LTPWHTNKNWSSVRVPTLIIGAENDTIAPVSSHAKPFY |
| | | NSLPSSTPKAYLELNGASHFAPNSSNTTIGKYSIAWLK |
| | | RFVDNDTRYSQFLCPAPHDDSAISEYRSTCPY |
| 22 | AncD3_S122P | AANPYERGPDPTESSLEASSGPFSVSETSVSRLSASGFG |
| | | GGTIYYPTTTSEGTYGAVAISPGYTATQSSIAWLGPRL |
| | | ASHGFVVITIDTNTTFDQPDSRARQLMAALNYLVNRSS |
| | | VRSRIDPSRLAVMGHSMGGGGTLRAAEDNPSLKAAIP |
| | | LTPWHTNKNWSSVRVPTLIIGAENDTIAPVSSHAKPFY |
| | | NSLPSSTPKAYLELNGASHFAPNSSNTTIGKYSIAWLK |
| | | RFVDNDTRYSQFLCPAPHDDSAISEYRSTCPY |
| 23 | AncD3_S214T | AANPYERGPDPTESSLEASSGPFSVSETSVSRLSASGFG |
| | | GGTIYYPTTTSEGTYGAVAISPGYTATQSSIAWLGPRL |
| | | ASHGFVVITIDTNTTFDQPDSRARQLMAALNYLVNRSS |
| | | VRSRIDSSRLAVMGHSMGGGGTLRAAEDNPSLKAAIP |
| | | LTPWHTNKNWSSVRVPTLIIGAENDTIAPVSSHAKPFY |
| | | NSLPSSTPKAYLELNGASHFAPNTSNTTIGKYSIAWLK |
| | | RFVDNDTRYSQFLCPAPHDDSAISEYRSTCPY |
| 24 | AncD3_A225S | AANPYERGPDPTESSLEASSGPFSVSETSVSRLSASGFG |
| | | GGTIYYPTTTSEGTYGAVAISPGYTATQSSIAWLGPRL |
| | | ASHGFVVITIDTNTTFDQPDSRARQLMAALNYLVNRSS |
| | | VRSRIDSSRLAVMGHSMGGGGTLRAAEDNPSLKAAIP |
| | | LTPWHTNKNWSSVRVPTLIIGAENDTIAPVSSHAKPFY |
| | | NSLPSSTPKAYLELNGASHFAPNSSNTTIGKYSISWLKR |
| | | FVDNDTRYSQFLCPAPHDDSAISEYRSTCPY |
| 25 | AncD3_S238T | AANPYERGPDPTESSLEASSGPFSVSETSVSRLSASGFG |
| | | GGTIYYPTTTSEGTYGAVAISPGYTATQSSIAWLGPRL |
| | | ASHGFVVITIDTNTTFDQPDSRARQLMAALNYLVNRSS |
| | | VRSRIDSSRLAVMGHSMGGGGTLRAAEDNPSLKAAIP |
| | | LTPWHTNKNWSSVRVPTLIIGAENDTIAPVSSHAKPFY |
| | | NSLPSSTPKAYLELNGASHFAPNSSNTTIGKYSIAWLK |
| | | RFVDNDTRYTQFLCPAPHDDSAISEYRSTCPY |
| 26 | AncD3_H246S | AANPYERGPDPTESSLEASSGPFSVSETSVSRLSASGFG |
| | | GGTIYYPTTTSEGTYGAVAISPGYTATQSSIAWLGPRL |
| | | ASHGFVVITIDTNTTFDQPDSRARQLMAALNYLVNRSS |
| | | VRSRIDSSRLAVMGHSMGGGGTLRAAEDNPSLKAAIP |
| | | LTPWHTNKNWSSVRVPTLIIGAENDTIAPVSSHAKPFY |
| | | NSLPSSTPKAYLELNGASHFAPNSSNTTIGKYSIAWLK |
| | | RFVDNDTRYSQFLCPAPSDDSAISEYRSTCPY |
| 27 | AncD3_S249A | AANPYERGPDPTESSLEASSGPFSVSETSVSRLSASGFG |
| | | GGTIYYPTTTSEGTYGAVAISPGYTATQSSIAWLGPRL |
| | | ASHGFVVITIDTNTTFDQPDSRARQLMAALNYLVNRSS |
| | | VRSRIDSSRLAVMGHSMGGGGTLRAAEDNPSLKAAIP |
| | | LTPWHTNKNWSSVRVPTLIIGAENDTIAPVSSHAKPFY |
| | | NSLPSSTPKAYLELNGASHFAPNSSNTTIGKYSIAWLK |
| | | RFVDNDTRYSQFLCPAPHDDAAISEYRSTCPY |
| 28 | N-terminal | AANP |
| | cellular export | |
| | signal peptide | |
| 29 | N-terminal | ADNP |
| | cellular export | |
| | signal peptide | |
| 30 | AncG4_N205C_ | AANPYERGPDPTEASLEASSGPFSVSETSVSRLSASGFG |
| | S252C_F210V | GGTIYYPTTTSSGTYGAVAISPGYTATQSSIAWLGPRLA |
| | | SHGFVVITIDTNTTFDQPDSRARQLMAALNYLVNRSSV |
| | | RSRIDSSRLAVMGHSMGGGGTLRAAEDNPSLKAAIPLT |
| | | PWHTNKNWSSVRVPTLIIGAENDTIAPVSSHAKPFYNS |
| | | LPSSTPKAYLELCGASHVAPNSSNTTIGKYSVSWLKRF |
| | | VDNDTRYSQFLCPAPHDDSAICEYRSTCPY |

1. A polypeptide having esterase activity, wherein the polypeptide comprises an amino acid sequence that has at least 80% identity to amino acids 2-257 of SEQ ID NO:1.

2-3. (canceled)

4. The polypeptide of claim 1, wherein the polypeptide comprises an amino acid sequence that differs from SEQ ID NO:1 by an amino acid substitution at one or more positions corresponding to amino acid position N202, F207 or S249 of SEQ ID NO:1.

5-10. (canceled)

11. The polypeptide of claim 4, wherein the polypeptide comprises an amino acid sequence that differs from SEQ ID NO:1 by amino acid substitutions at positions corresponding to amino acid positions N202, F207 and S249 of SEQ ID NO:1.

12. (canceled)

13. The polypeptide of claim 1, wherein the polypeptide comprises an amino acid sequence that differs from SEQ ID NO:1 by an amino acid substitution at one or more positions corresponding to amino acid position D7, E10, A11, L13, E24, A32, T45, S48, Y51, P72, N87, F90, N105, S119, N158, S192, S211, V221, S222, S235, H243 or S246 of SEQ ID NO:1.

14-27. (canceled)

28. The polypeptide of claim 13, wherein the polypeptide comprises an amino acid sequence that differs from SEQ ID NO:1 by amino acid substitutions at positions corresponding to amino acid positions A11, S48 and V221 of SEQ ID NO:1.

29. (canceled)

30. The polypeptide of claim 13, wherein the polypeptide comprises an amino acid sequence that differs from SEQ ID NO:1 by amino acid substitutions at positions corresponding to amino acid positions A11, V221 and S222 of SEQ ID NO:1.

31. (canceled)

32. The polypeptide of claim 13, wherein the polypeptide comprises an amino acid sequence that differs from SEQ ID NO:1 by amino acid substitutions at positions corresponding to amino acid positions A11, S48, V221 and S222 of SEQ ID NO:1.

33. (canceled)

34. The polypeptide of claim 1, wherein the polypeptide comprises an amino acid sequence that differs from SEQ ID NO:1 by an additional amino acid substitution at a position corresponding to amino acid position E10, E24, A32, T45, Y51, P72, F90, N105, S119, S211, S235, H243 or S246 of SEQ ID NO:1.

35-63. (canceled)

64. The polypeptide of claim 1, wherein the polypeptide comprises an amino acid sequence comprising SEQ ID NOs:2-27 or 30.

65. (canceled)

66. A polynucleotide comprising the nucleic acid sequence encoding the polypeptide of claim 1.

67-68. (canceled)

69. A host cell comprising the polynucleotide of claim 10.

70. A method of producing a polypeptide having esterase activity, the method comprising
a. providing the polynucleotide of claim 66;
b. expressing the polynucleotide in a host cell under conditions sufficient to allow the host cell to produce the polypeptide by the host cell; and
c. collecting the polypeptide produced by the host cell in (b).

71. A method for hydrolysing a terephthalic acid ester, the method comprising contacting a terephthalic acid ester with the polypeptide of claim 1, under conditions sufficient to convert the terephthalic acid ester to terephthalic acid and ethylene glycol, mono-(2-hydroxyethyl) terephthalate and/or bis-(2-hydroxyethyl) terephthalate.

72-73. (canceled)

74. A method of degrading a plastic product comprising a polyester, the method comprising contacting the plastic product to the polypeptide of claim 1.

75. The method of claim 74, wherein the polyester is selected from the group consisting of polylactic acid (PLA), polytrimethylene terephthalate (PTT), polybutylene terephthalate (PBT), polyethylene isosorbide terephthalate (PEIT), polyethylene terephthalate (PET), polyhydroxyalkanoate (PHA), polybutylene succinate (PBS), polybutylene succinate adipate (PBSA), polybutylene adipate terephthalate (PBAT), polyethylene furanoate (PEF), polycapro lactone (PCL), polyethylene adipate (PEA), poly(glycolic acid) (PGA), poly(lactic-co-glycolic acid) (PLGA) and combinations of any of the foregoing.

76. The method of claim 75, wherein the polyester is polyethylene terephthalate (PET).

77. The method of claim 76, comprising contacting the PET to the polypeptide of claim 1, under conditions sufficient to generate terephthalic acid and ethylene glycol, mono-(2-hydroxyethyl) terephthalate and/or bis-(2-hydroxyethyl) terephthalate.

78. The method of claim 77, further comprising simultaneously or sequentially exposing the PET to
a. a mono-(2-hydroxyethyl) terephthalate hydrolase (MHETase), or an enzyme having MHETase activity;
b. a wild-type or extant PETase; and/or
c. a cutinase having PETase activity.

79. The method of claim 78, further comprising recovering the generated terephthalic acid, ethylene glycol, mono-(2-hydroxyethyl) terephthalate and/or bis-(2-hydroxyethyl) terephthalate.

80. A composition comprising the terephthalic acid, the ethylene glycol, the mono-(2-hydroxyethyl) terephthalate and/or the bis-(2-hydroxyethyl) terephthalate recovered by the method of claim 79.

81-82. (canceled)

83. The method of claim 76, comprising contacting the PET to the host cell of claim 69, under conditions sufficient to generate terephthalic acid and ethylene glycol, mono-(2-hydroxyethyl) terephthalate and/or bis-(2-hydroxyethyl) terephthalate.


SEQUENCE LISTING (30 sequences)

SEQ ID NO: 1 - protein, 257 aa, synthetic construct
MYERGPDPTEASLEASSGPFSVSETSVSRLSASGFGGGTIYYPTTTSSGTYGAVAISPGYTATQSSIAWLGPRLASHGFVVITIDTNTTFDQPDSRARQLMAALNYLVNRSSVRSRIDSSRLAVMGHSMGGGGTLRAAEDNPSLKAAIPLTPWHTNKNWSSVRVPTLIIGAENDTIAPVSSHAKPFYNSLPSSTPKAYLELNGASHFAPNSSNTTIGKYSVSWLKRFVDNDTRYSQFLCPAPHDDSAISEYRSTCPY

SEQ ID NO: 2 - protein, 257 aa, synthetic construct
MYERGPDPTESSLEASSGPFSVSETSVSRLSASGFGGGTIYYPTTTSEGTYGAVAISPGYTATQSSIAWLGPRLASHGFVVITIDTNTTFDQPDSRARQLMAALNYLVNRSSVRSRIDSSRLAVMGHSMGGGGTLRAAEDNPSLKAAIPLTPWHTNKNWSSVRVPTLIIGAENDTIAPVSSHAKPFYNSLPSSTPKAYLELNGASHFAPNSSNTTIGKYSIAWLKRFVDNDTRYSQFLCPAPHDDSAISEYRSTCPY

SEQ ID NO: 3 - protein, 260 aa, synthetic construct
AANPYERGPDPTEASLEASSGPFSVSETSVSRLSASGFGGGTIYYPTTTSSGTYGAVAISPGYTATQSSIAWLGPRLASHGFVVITIDTNTTFDQPDSRARQLMAALNYLVNRSSVRSRIDSSRLAVMGHSMGGGGTLRAAEDNPSLKAAIPLTPWHTNKNWSSVRVPTLIIGAENDTIAPVSSHAKPFYNSLPSSTPKAYLELNGASHFAPNSSNTTIGKYSVSWLKRFVDNDTRYSQFLCPAPHDDSAISEYRSTCPY

SEQ ID NO: 4 - protein, 260 aa, synthetic construct
AANPYERGPDPTESSLEASSGPFSVSETSVSRLSASGFGGGTIYYPTTTSEGTYGAVAISPGYTATQSSIAWLGPRLASHGFVVITIDTNTTFDQPDSRARQLMAALNYLVNRSSVRSRIDSSRLAVMGHSMGGGGTLRAAEDNPSLKAAIPLTPWHTNKNWSSVRVPTLIIGAENDTIAPVSSHAKPFYNSLPSSTPKAYLELNGASHFAPNSSNTTIGKYSIAWLKRFVDNDTRYSQFLCPAPHDDSAISEYRSTCPY

SEQ ID NO: 5 - protein, 260 aa, synthetic construct
ADNPYERGPDPTEASLEASSGPFSVSETSVSRLSASGFGGGTIYYPTTTSSGTYGAVAISPGYTATQSSIAWLGPRLASHGFVVITIDTNTTFDQPDSRARQLMAALNYLVNRSSVRSRIDSSRLAVMGHSMGGGGTLRAAEDNPSLKAAIPLTPWHTNKNWSSVRVPTLIIGAENDTIAPVSSHAKPFYNSLPSSTPKAYLELNGASHFAPNSSNTTIGKYSVSWLKRFVDNDTRYSQFLCPAPHDDSAISEYRSTCPY

SEQ ID NO: 6 - protein, 260 aa, synthetic construct
AANPYERGPEPTEASLEASSGPFSVSETSVSRLSASGFGGGTIYYPTTTSSGTYGAVAISPGYTATQSSIAWLGPRLASHGFVVITIDTNTTFDQPDSRARQLMAALNYLVNRSSVRSRIDSSRLAVMGHSMGGGGTLRAAEDNPSLKAAIPLTPWHTNKNWSSVRVPTLIIGAENDTIAPVSSHAKPFYNSLPSSTPKAYLELNGASHFAPNSSNTTIGKYSVSWLKRFVDNDTRYSQFLCPAPHDDSAISEYRSTCPY

SEQ ID NO: 7 - protein, 260 aa, synthetic construct
AANPYERGPDPTEASVEASSGPFSVSETSVSRLSASGFGGGTIYYPTTTSSGTYGAVAISPGYTATQSSIAWLGPRLASHGFVVITIDTNTTFDQPDSRARQLMAALNYLVNRSSVRSRIDSSRLAVMGHSMGGGGTLRAAEDNPSLKAAIPLTPWHTNKNWSSVRVPTLIIGAENDTIAPVSSHAKPFYNSLPSSTPKAYLELNGASHFAPNSSNTTIGKYSVSWLKRFVDNDTRYSQFLCPAPHDDSAISEYRSTCPY

SEQ ID NO: 8 - protein, 260 aa, synthetic construct
AANPYERGPDPTEASLEASSGPFSVSETSVSRLSASGFGGGTIYYPTTTSAGTYGAVAISPGYTATQSSIAWLGPRLASHGFVVITIDTNTTFDQPDSRARQLMAALNYLVNRSSVRSRIDSSRLAVMGHSMGGGGTLRAAEDNPSLKAAIPLTPWHTNKNWSSVRVPTLIIGAENDTIAPVSSHAKPFYNSLPSSTPKAYLELNGASHFAPNSSNTTIGKYSVSWLKRFVDNDTRYSQFLCPAPHDDSAISEYRSTCPY

SEQ ID NO: 9 - protein, 260 aa, synthetic construct
AANPYERGPDPTEASLEASSGPFSVSETSVSRLSASGFGGGTIYYPTTTSSGTYGAVAISPGYTATQSSIAWLGPRLASHGFVVITIDTQTTFDQPDSRARQLMAALNYLVNRSSVRSRIDSSRLAVMGHSMGGGGTLRAAEDNPSLKAAIPLTPWHTNKNWSSVRVPTLIIGAENDTIAPVSSHAKPFYNSLPSSTPKAYLELNGASHFAPNSSNTTIGKYSVSWLKRFVDNDTRYSQFLCPAPHDDSAISEYRSTCPY

SEQ ID NO: 10 - protein, 260 aa, synthetic construct
AANPYERGPDPTEASLEASSGPFSVSETSVSRLSASGFGGGTIYYPTTTSSGTYGAVAISPGYTATQSSIAWLGPRLASHGFVVITIDTNTTFDQPDSRARQLMAALNYLVNRSSVRSRIDSSRLAVMGHSMGGGGTLRAAEDNPSLKAAIPLTPWHTNKDWSSVRVPTLIIGAENDTIAPVSSHAKPFYNSLPSSTPKAYLELNGASHFAPNSSNTTIGKYSVSWLKRFVDNDTRYSQFLCPAPHDDSAISEYRSTCPY

SEQ ID NO: 11 - protein, 260 aa, synthetic construct
AANPYERGPDPTEASLEASSGPFSVSETSVSRLSASGFGGGTIYYPTTTSSGTYGAVAISPGYTATQSSIAWLGPRLASHGFVVITIDTNTTFDQPDSRARQLMAALNYLVNRSSVRSRIDSSRLAVMGHSMGGGGTLRAAEDNPSLKAAIPLTPWHTNKNWSSVRVPTLIIGAENDTIAPVSSHAKPFYNSLPASTPKAYLELNGASHFAPNSSNTTIGKYSVSWLKRFVDNDTRYSQFLCPAPHDDSAISEYRSTCPY

SEQ ID NO: 12 - protein, 260 aa, synthetic construct
AANPYERGPDPTEASLEASSGPFSVSETSVSRLSASGFGGGTIYYPTTTSSGTYGAVAISPGYTATQSSIAWLGPRLASHGFVVITIDTNTTFDQPDSRARQLMAALNYLVNRSSVRSRIDSSRLAVMGHSMGGGGTLRAAEDNPSLKAAIPLTPWHTNKNWSSVRVPTLIIGAENDTIAPVSSHAKPFYNSLPSSTPKAYLELNGASHFAPNSSNTTIGKYSVSWLKRFVDNDTRYNQFLCPAPHDDSAISEYRSTCPY

SEQ ID NO: 13 - protein, 260 aa, synthetic construct
AANPYERGPDPTDSSLEASSGPFSVSETSVSRLSASGFGGGTIYYPTTTSEGTYGAVAISPGYTATQSSIAWLGPRLASHGFVVITIDTNTTFDQPDSRARQLMAALNYLVNRSSVRSRIDSSRLAVMGHSMGGGGTLRAAEDNPSLKAAIPLTPWHTNKNWSSVRVPTLIIGAENDTIAPVSSHAKPFYNSLPSSTPKAYLELNGASHFAPNSSNTTIGKYSIAWLKRFVDNDTRYSQFLCPAPHDDSAISEYRSTCPY

SEQ ID NO: 14 - protein, 260 aa, synthetic construct
AANPYERGPDPTESSLEASSGPFSVSQTSVSRLSASGFGGGTIYYPTTTSEGTYGAVAISPGYTATQSSIAWLGPRLASHGFVVITIDTNTTFDQPDSRARQLMAALNYLVNRSSVRSRIDSSRLAVMGHSMGGGGTLRAAEDNPSLKAAIPLTPWHTNKNWSSVRVPTLIIGAENDTIAPVSSHAKPFYNSLPSSTPKAYLELNGASHFAPNSSNTTIGKYSIAWLKRFVDNDTRYSQFLCPAPHDDSAISEYRSTCPY

SEQ ID NO: 15 - protein, 260 aa, synthetic construct
AANPYERGPDPTESSLEASSGPFSVSETSVSRLSVSGFGGGTIYYPTTTSEGTYGAVAISPGYTATQSSIAWLGPRLASHGFVVITIDTNTTFDQPDSRARQLMAALNYLVNRSSVRSRIDSSRLAVMGHSMGGGGTLRAAEDNPSLKAAIPLTPWHTNKNWSSVRVPTLIIGAENDTIAPVSSHAKPFYNSLPSSTPKAYLELNGASHFAPNSSNTTIGKYSIAWLKRFVDNDTRYSQFLCPAPHDDSAISEYRSTCPY

SEQ ID NO: 16 - protein, 260 aa, synthetic construct
AANPYERGPDPTESSLEASSGPFSVSETSVSRLSASGFGGGTIYYPTSTSEGTYGAVAISPGYTATQSSIAWLGPRLASHGFVVITIDTNTTFDQPDSRARQLMAALNYLVNRSSVRSRIDSSRLAVMGHSMGGGGTLRAAEDNPSLKAAIPLTPWHTNKNWSSVRVPTLIIGAENDTIAPVSSHAKPFYNSLPSSTPKAYLELNGASHFAPNSSNTTIGKYSIAWLKRFVDNDTRYSQFLCPAPHDDSAISEYRSTCPY

SEQ ID NO: 17 - protein, 260 aa, synthetic construct
AANPYERGPDPTESSLEASSGPFSVSETSVSRLSASGFGGGTIYYPTTTSSGTYGAVAISPGYTATQSSIAWLGPRLASHGFVVITIDTNTTFDQPDSRARQLMAALNYLVNRSSVRSRIDSSRLAVMGHSMGGGGTLRAAEDNPSLKAAIPLTPWHTNKNWSSVRVPTLIIGAENDTIAPVSSHAKPFYNSLPSSTPKAYLELNGASHFAPNSSNTTIGKYSIAWLKRFVDNDTRYSQFLCPAPHDDSAISEYRSTCPY

SEQ ID NO: 18 - protein, 260 aa, synthetic construct
AANPYERGPDPTESSLEASSGPFSVSETSVSRLSASGFGGGTIYYPTTTSEGTFGAVAISPGYTATQSSIAWLGPRLASHGFVVITIDTNTTFDQPDSRARQLMAALNYLVNRSSVRSRIDSSRLAVMGHSMGGGGTLRAAEDNPSLKAAIPLTPWHTNKNWSSVRVPTLIIGAENDTIAPVSSHAKPFYNSLPSSTPKAYLELNGASHFAPNSSNTTIGKYSIAWLKRFVDNDTRYSQFLCPAPHDDSAISEYRSTCPY

SEQ ID NO: 19 - protein, 260 aa, synthetic construct
AANPYERGPDPTESSLEASSGPFSVSETSVSRLSASGFGGGTIYYPTTTSEGTYGAVAISPGYTATQSSIAWLGRRLASHGFVVITIDTNTTFDQPDSRARQLMAALNYLVNRSSVRSRIDSSRLAVMGHSMGGGGTLRAAEDNPSLKAAIPLTPWHTNKNWSSVRVPTLIIGAENDTIAPVSSHAKPFYNSLPSSTPKAYLELNGASHFAPNSSNTTIGKYSIAWLKRFVDNDTRYSQFLCPAPHDDSAISEYRSTCPY

SEQ ID NO: 20 - protein, 260 aa, synthetic construct
AANPYERGPDPTESSLEASSGPFSVSETSVSRLSASGFGGGTIYYPTTTSEGTYGAVAISPGYTATQSSIAWLGPRLASHGFVVITIDTNTTLDQPDSRARQLMAALNYLVNRSSVRSRIDSSRLAVMGHSMGGGGTLRAAEDNPSLKAAIPLTPWHTNKNWSSVRVPTLIIGAENDTIAPVSSHAKPFYNSLPSSTPKAYLELNGASHFAPNSSNTTIGKYSIAWLKRFVDNDTRYSQFLCPAPHDDSAISEYRSTCPY

SEQ ID NO: 21 - protein, 260 aa, synthetic construct
AANPYERGPDPTESSLEASSGPFSVSETSVSRLSASGFGGGTIYYPTTTSEGTYGAVAISPGYTATQSSIAWLGPRLASHGFVVITIDTNTTFDQPDSRARQLMAALDYLVNRSSVRSRIDSSRLAVMGHSMGGGGTLRAAEDNPSLKAAIPLTPWHTNKNWSSVRVPTLIIGAENDTIAPVSSHAKPFYNSLPSSTPKAYLELNGASHFAPNSSNTTIGKYSIAWLKRFVDNDTRYSQFLCPAPHDDSAISEYRSTCPY

SEQ ID NO: 22 - protein, 260 aa, synthetic construct
AANPYERGPDPTESSLEASSGPFSVSETSVSRLSASGFGGGTIYYPTTTSEGTYGAVAISPGYTATQSSIAWLGPRLASHGFVVITIDTNTTFDQPDSRARQLMAALNYLVNRSSVRSRIDPSRLAVMGHSMGGGGTLRAAEDNPSLKAAIPLTPWHTNKNWSSVRVPTLIIGAENDTIAPVSSHAKPFYNSLPSSTPKAYLELNGASHFAPNSSNTTIGKYSIAWLKRFVDNDTRYSQFLCPAPHDDSAISEYRSTCPY

SEQ ID NO: 23 - protein, 260 aa, synthetic construct
AANPYERGPDPTESSLEASSGPFSVSETSVSRLSASGFGGGTIYYPTTTSEGTYGAVAISPGYTATQSSIAWLGPRLASHGFVVITIDTNTTFDQPDSRARQLMAALNYLVNRSSVRSRIDSSRLAVMGHSMGGGGTLRAAEDNPSLKAAIPLTPWHTNKNWSSVRVPTLIIGAENDTIAPVSSHAKPFYNSLPSSTPKAYLELNGASHFAPNTSNTTIGKYSIAWLKRFVDNDTRYSQFLCPAPHDDSAISEYRSTCPY

SEQ ID NO: 24 - protein, 260 aa, synthetic construct
AANPYERGPDPTESSLEASSGPFSVSETSVSRLSASGFGGGTIYYPTTTSEGTYGAVAISPGYTATQSSIAWLGPRLASHGFVVITIDTNTTFDQPDSRARQLMAALNYLVNRSSVRSRIDSSRLAVMGHSMGGGGTLRAAEDNPSLKAAIPLTPWHTNKNWSSVRVPTLIIGAENDTIAPVSSHAKPFYNSLPSSTPKAYLELNGASHFAPNSSNTTIGKYSISWLKRFVDNDTRYSQFLCPAPHDDSAISEYRSTCPY

SEQ ID NO: 25 - protein, 260 aa, synthetic construct
AANPYERGPDPTESSLEASSGPFSVSETSVSRLSASGFGGGTIYYPTTTSEGTYGAVAISPGYTATQSSIAWLGPRLASHGFVVITIDTNTTFDQPDSRARQLMAALNYLVNRSSVRSRIDSSRLAVMGHSMGGGGTLRAAEDNPSLKAAIPLTPWHTNKNWSSVRVPTLIIGAENDTIAPVSSHAKPFYNSLPSSTPKAYLELNGASHFAPNSSNTTIGKYSIAWLKRFVDNDTRYTQFLCPAPHDDSAISEYRSTCPY

SEQ ID NO: 26 - protein, 260 aa, synthetic construct
AANPYERGPDPTESSLEASSGPFSVSETSVSRLSASGFGGGTIYYPTTTSEGTYGAVAISPGYTATQSSIAWLGPRLASHGFVVITIDTNTTFDQPDSRARQLMAALNYLVNRSSVRSRIDSSRLAVMGHSMGGGGTLRAAEDNPSLKAAIPLTPWHTNKNWSSVRVPTLIIGAENDTIAPVSSHAKPFYNSLPSSTPKAYLELNGASHFAPNSSNTTIGKYSIAWLKRFVDNDTRYSQFLCPAPSDDSAISEYRSTCPY

SEQ ID NO: 27 - protein, 260 aa, synthetic construct
AANPYERGPDPTESSLEASSGPFSVSETSVSRLSASGFGGGTIYYPTTTSEGTYGAVAISPGYTATQSSIAWLGPRLASHGFVVITIDTNTTFDQPDSRARQLMAALNYLVNRSSVRSRIDSSRLAVMGHSMGGGGTLRAAEDNPSLKAAIPLTPWHTNKNWSSVRVPTLIIGAENDTIAPVSSHAKPFYNSLPSSTPKAYLELNGASHFAPNSSNTTIGKYSIAWLKRFVDNDTRYSQFLCPAPHDDAAISEYRSTCPY

SEQ ID NO: 28 - protein, 4 aa, synthetic construct
AANP

SEQ ID NO: 29 - protein, 4 aa, synthetic construct
ADNP

SEQ ID NO: 30 - protein, 260 aa, synthetic construct
AANPYERGPDPTEASLEASSGPFSVSETSVSRLSASGFGGGTIYYPTTTSSGTYGAVAISPGYTATQSSIAWLGPRLASHGFVVITIDTNTTFDQPDSRARQLMAALNYLVNRSSVRSRIDSSRLAVMGHSMGGGGTLRAAEDNPSLKAAIPLTPWHTNKNWSSVRVPTLIIGAENDTIAPVSSHAKPFYNSLPSSTPKAYLELCGASHVAPNSSNTTIGKYSVSWLKRFVDNDTRYSQFLCPAPHDDSAICEYRSTCPY